(19) United States
(12) Patent Application Publication
(10) Pub. No.: US 2020/0006668 A1
(43) Pub. Date: Jan. 2, 2020

(54) FUSED CYCLIC COMPOUND, AND PREPARATION METHOD AND USE THEREOF

(71) Applicant: Ningbo Lumilan Advanced Materials., Ltd., Ningbo City (CN)

(72) Inventors: Hua SUN, Ningbo City (CN); Zhi Kuan CHEN, Ningbo City (CN)

(21) Appl. No.: 16/262,046

(22) Filed: Jan. 30, 2019

(30) Foreign Application Priority Data
Jun. 28, 2018 (CN) 201810690615.X

(51) Int. Cl. H01L 51/00; C09K 11/06; C07D 491/22; C07D 487/22; C07D 491/16; C07D 487/16

(52) U.S. Cl. CPC H01L 51/0072; C09K 11/06; C07D 491/22; H01L 51/0071; H01L 51/5016; C07D 487/22; C07D 491/16; C07D 487/16; H01L 2251/552; H01L 51/0067

(57) ABSTRACT

The invention relates to a fused cyclic compound having a structure of Formula in the fused cyclic compound by controlling effective conjugation of aromatic and heterocyclic rings. Electron transport performance is balanced while hole performance of fused cyclic compound improves. The compound has high triplet energy level and glass transition temperature. The material molecule isn't prone to crystallization. The compound ensures transfer of energy to a guest material. Adjusting substituents of fused cyclic compound improves electron and hole transport performances, reduces difference between singlet and triplet energy levels, broadens recombination region of carriers, and prevents triplet-triplet exciton annihilation. An organic light-emitting device contains the fused cyclic compound. The compound is used as a host material in a light emitting layer. The energy level of the light emitting layer becomes more matched with adjacent carrier transport layers, reducing driving voltage of the device while increasing the luminescence efficiency of the device.

### CROSS-REFERENCE TO RELATED APPLICATION

This application claims the priority to Chinese Patent Application No. 201810690615.X filed on Jun. 28, 2018 with the State Intellectual Property Office of the People's Republic of China, and entitled “Fused cyclic compound and preparation method and use thereof”, which is incorporated herein by reference in its entirety.

### TECHNICAL FIELD

The present invention relates to the field of display technologies, and particularly to a fused cyclic compound, and a preparation method and use thereof.

### RELATED ART

Pope et al. initially discovered the electroluminescence properties of single crystal anthracene in 1965. This is the first electroluminescence phenomenon of organic compounds. In 1987, a low-voltage high-brightness organic light-emitting diode (OLED) was successfully developed by Tang et al. from Kodak Company (US), using an organic small molecular semiconductor material. As a new display technology, the organic light-emitting diode (OLED) has many advantages such as self-lighting, wide viewing angle, low power consumption, rich color, fast response time, applicability over wide temperature range and flexible display, thus having broad application prospects in the field of display and illumination, and receiving more and more attention.

OLEDs often have a sandwich structure, that is, an organic light-emitting layer is sandwiched between electrodes at two sides. The light-emission mechanism is as follows. Driven by an external electric field, electrons and holes are injected into an organic electron transport layer and a hole transport layer respectively from a cathode and an anode, and recombined to form excitons in an organic light-emitting layer, and then the excitons go back to the ground state by radiation transition and emit light. In the electroluminescence process, singlet and triplet excitons are simultaneously generated. According to the statistics of electron spin, the ratio of singlet excitons to triplet excitons is inferred to be 1:3. When the singlet excitons transition back to the ground state, the material fluoresces, and when the triplet excitons transition back to the ground state, and the material phosphoresces.

Fluorescent materials are the initially used organic electroluminescent materials, which are diverse and inexpensive. However, due to the limitation by spin-forbidden transition of the electrons, only 25% of the singlet excitons can be utilized for emitting light, so the internal quantum efficiency is low, which limits the efficiency of the device. For phosphorescent materials, by means of the spin coupling of heavy atoms, the energy of singlet excitons is allowed to be transferred to triplet excitons by intersystem crossing (ISC), and then triplet excitons phosphoresce. Accordingly, an internal quantum efficiency of 100% is theoretically achievable. However, concentration quenching and triplet-triplet annihilation are common in phosphorescent devices, which affects the luminescence efficiency of the devices.

The OLED devices fabricated by the doping method are advantageous in the luminescence efficiency of the devices. Therefore, the material of the light-emitting layer is often formed by doping a guest material into a host material, where the host material is an important factor affecting the luminescence efficiency and performance of the OLED devices. 4,4′-Bis(9H-carbazol-9-yl)biphenyl (CBP) is a widely used host material with good hole transport performance. However, when CBP is used as a host material, the molecular packing state and the film morphology are prone to change and the molecules tend to recrystallize under working conditions due to the low glass transition temperature of CBP, causing deceased performance and luminescence efficiency of the OLED device during use. Moreover, CBP is a hole-type host material, the transport of electrons and holes is unbalanced, the efficiency of recombination into excitons is low, the light-emitting region is not desirable, the roll-off phenomenon of the device is serious during operation, and the triplet energy of CBP is lower than that of the blue dopant material, resulting in a low efficiency of energy transfer from the host material to the guest material, thus reducing the efficiency of the devices.

### SUMMARY

Therefore, the technical problems to be solved by the present invention are to overcome the disadvantages in the prior art of low triplet energy level and tendency to crystallization of the host material in the light-emitting layer, as well as unbalanced charge transport of the host material, undesirable light-emitting region, and inability of efficiently transporting the energy of the host material to the guest material, resulting in low luminescence efficiency and performance of the devices.

To this end, the present invention provides the following technical solutions.

In a first aspect, the present invention provides a fused cyclic compound, having a structure of Formula (I) or (II):

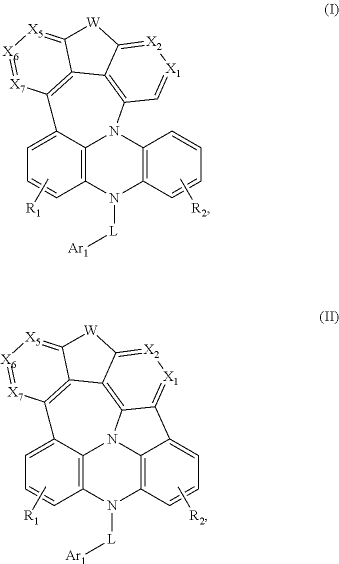

wherein W is selected from O, S, C—Ar₂ or N—Ar₂;

X₁ is selected from N or C—R₁ₐ, X₂ is selected from N or C—R₂ₐ, X₅ is selected from N or C—R₅ₐ, X₆ is selected from N or C—R₆ₐ, X₇ is selected from N or C—R₇ₐ, in which

R₁ₐ, R₂ₐ, R₅ₐ, R₆ₐ, and R₇ₐ are each independently selected from hydrogen, halo, cyano, alkyl, alkenyl, alkynyl, cycloalkyl, alkoxy, silyl, aryl or heteroaryl;

R₁, and R₂ are each independently selected from hydrogen, halo, cyano, alkyl, alkenyl, alkynyl, cycloalkyl, alkoxy, silyl, aryl or heteroaryl; L is a single bond, a C₁-C₁₀ substituted or unsubstituted aliphatic hydrocarbon group, a C₆-C₆₀ substituted or unsubstituted aryl group, or a C₃-C₃₀ substituted or unsubstituted heteroaryl group; Ar₁ and Ar₂ are each independently selected from hydrogen, halo, cyano, alkyl, alkenyl, alkynyl, cycloalkyl, alkoxy, silyl, aryl or heteroaryl, in which

the heteroaryl has at least one heteroatom independently selected from nitrogen, sulfur, oxygen, phosphorous, boron or silicon.

Preferably, in the fused cyclic compound,

R₁ and R₂ are each independently selected from hydrogen, halo, cyano, a C₁-C₃₀ substituted or unsubstituted alkyl group, a C₂-C₃₀ substituted or unsubstituted alkenyl group, a C₂-C₃₀ substituted or unsubstituted alkynyl group, a C₃-C₃₀ substituted or unsubstituted cycloalkyl group, a C₁-C₃₀ substituted or unsubstituted alkoxy group, a C₁-C₃₀ substituted or unsubstituted silyl group, a C₆-C₆₀ substituted or unsubstituted aryl group, or a C₃-C₃₀ substituted or unsubstituted heteroaryl group;

Ar₁ and Ar₂ are each independently selected from hydrogen, halo, cyano, a C₁-C₃₀ substituted or unsubstituted alkyl group, a C₂-C₃₀ substituted or unsubstituted alkenyl group, a C₂-C₃₀ substituted or unsubstituted alkynyl group, a C₃-C₃₀ substituted or unsubstituted cycloalkyl group, a C₁-C₃₀ substituted or unsubstituted alkoxy group, a C₁-C₃₀ substituted or unsubstituted silyl group, a C₆-C₆₀ substituted or unsubstituted aryl group, or a C₃-C₃₀ substituted or unsubstituted heteroaryl group; and

R₁ₐ, R₂ₐ, R₅ₐ, R₆ₐ, and R₇ₐ are each independently selected from hydrogen, halo, cyano, a C₁-C₃₀ substituted or unsubstituted alkyl group, a C₂-C₃₀ substituted or unsubstituted alkenyl group, a C₂-C₃₀ substituted or unsubstituted alkynyl group, a C₃-C₃₀ substituted or unsubstituted cycloalkyl group, a C₁-C₃₀ substituted or unsubstituted alkoxy group, a C₁-C₃₀ substituted or unsubstituted silyl group, a C₆-C₆₀ substituted or unsubstituted aryl group, or a C₃-C₃₀ substituted or unsubstituted heteroaryl group.

Preferably, in the fused cyclic compound, Ar₁ and Ar₂ are each independently selected from any of the following groups; and R₁, R₂, R₁ₐ, R₂ₐ, R₃ₐ, R₅ₐ, R₇ₐ are each independently selected from hydrogen or any of the following groups:

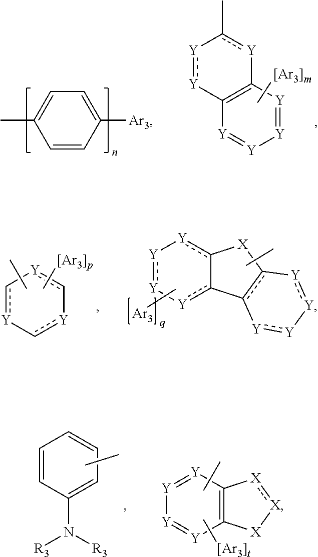

where X is nitrogen, oxygen or sulfur, Y is each independently nitrogen or carbon; and in the

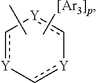

at least one Y is nitrogen; and

n is an integer of 0-5, m is an integer of 0-7, p is an integer of 0-6, q is an integer of 0-8, and t is an integer of 0-7; and [symbol] is a single or double bond;

R₃ is each independently selected from a substituted or unsubstituted phenyl group or hydrogen; and

Ar₃ is each independently selected from hydrogen, phenyl, coronenyl, pentalenyl, indenyl, naphthyl, azulenyl, fluorenyl, heptalenyl, octalenyl, benzodiindenyl, acenaphthylenyl, phenalenyl, phenanthrenyl, anthracenyl, triindenyl, fluoranthenyl, benzopyrenyl, benzoperylenyl, benzofluoranthenyl, acephenanthrenyl, aceanthrylenyl, 9,10-benzophenanthrenyl, pyrenyl, 1,2-benzophenanthrenyl, butylphenyl, naphthacenyl, pleiadenyl, picenyl, perylenyl, pentaphenyl, pentacenyl, tetraphenylene, cholanthrenyl, helicenyl, hexaphenyl, rubicenyl, coronenyl, trinaphthylenyl, heptaphenyl, pyranthrenyl, ovalenyl, corannulenyl, anthanthrenyl, truxenyl, pyranyl, benzopyranyl, furyl, benzofuryl, isobenzofuryl, oxanthracenyl, oxazolinyl, dibenzofuryl, peri-xanthenoxanthenyl, thienyl, thioxanthenyl, thianthrenyl, phenoxathiinyl, thionaphthenyl, isothionaphthenyl, thiophanthrenyl, dibenzothienyl, benzothienyl, pyrrolyl, pyrazolyl, tellurazolyl, selenazolyl, thiazolyl, isothiazolyl, oxazolyl, oxadiazolyl, furazanyl, pyridinyl, pyrazinyl, pyrimidinyl, pyridazinyl, triazinyl, indolizinyl, indolyl, isoindolyl, indazolyl, purinyl, quinolizinyl, isoquinolyl, carbazolyl, fluorenocarbazolyl, indolocarbazolyl, imidazolyl, naphthyridinyl, phthalazinyl, quinazolinyl, benzodiazepinyl, quinoxalinyl, cinnolinyl, quinolyl, pteridinyl, phenanthridinyl, acridinyl, perimidinyl, phenanthrolinyl, phenazinyl, carbolinyl, phenotellurazinyl, phenoselenazinyl, phenothiazinyl, phenoxazinyl, triphenodithiazinyl, azadibenzofuryl, triphenodioxazinyl, anthrazinyl, benzothiazolyl, benzoimidazolyl, benzoxazolyl, benzisoxazolyl or benzoisothiazolyl.

Preferably, in the fused cyclic compound, R₁, R₂ and Ar₁ include at least one electron withdrawing group, and/or at least one electron donating group.

Preferably, the fused cyclic compound having a molecular structure shown below:

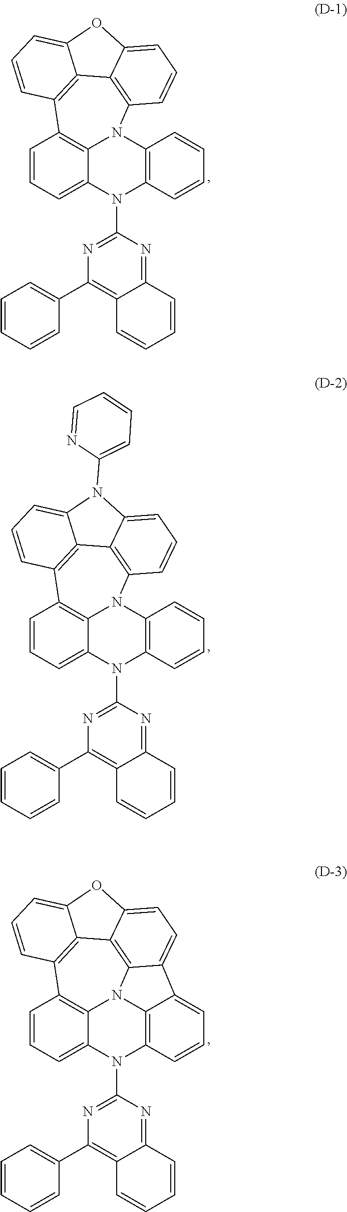 [SMILES: c1ccc(-c2nc(N3c4ccccc4-n4c5cccc6oc7cccc(c8cccc3c84)c7c65)nc3ccccc23)cc1 ; c1ccc(-c2nc(N3c4ccccc4-n4c5cccc6c5c5c(cccc5n6-c5ccccn5)c5cccc3c54)nc3ccccc23)cc1 ; c1ccc(-c2nc(N3c4cccc5c6cccc7oc8ccc9c%10cccc3c%10n(c45)c9c8c76)nc3ccccc23)cc1]

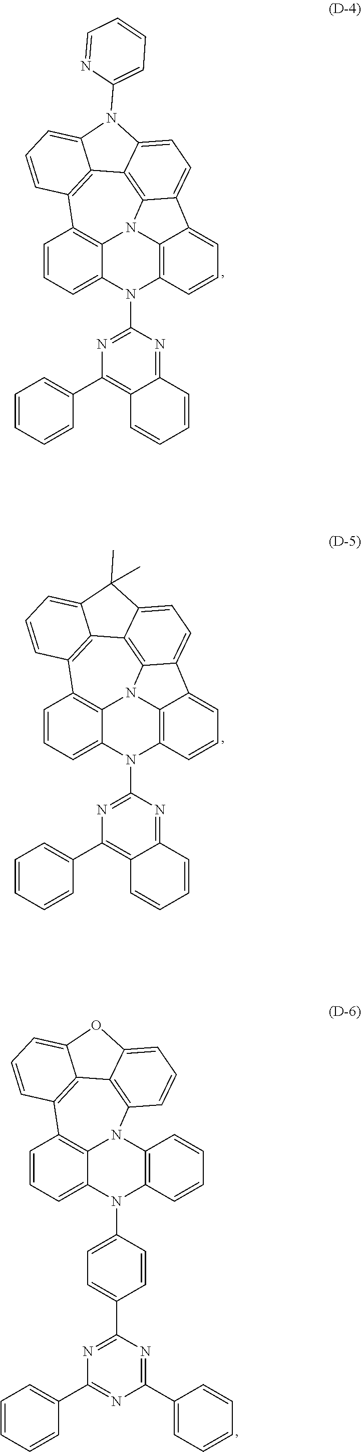 [SMILES: c1ccc(-c2nc(N3c4cccc5c6cccc7c6c6c(ccc8c9cccc3c9n(c45)c86)n7-c3ccccn3)nc3ccccc23)cc1 ; CC1(C)c2cccc3c2-c2c1ccc1c4cccc5c4n(c21)-c1c-3cccc1N5c1nc(-c2ccccc2)c2ccccc2n1 ; c1ccc(-c2nc(-c3ccccc3)nc(-c3ccc(N4c5ccccc5-n5c6cccc7oc8cccc(c9cccc4c95)c8c76)cc3)n2)cc1]

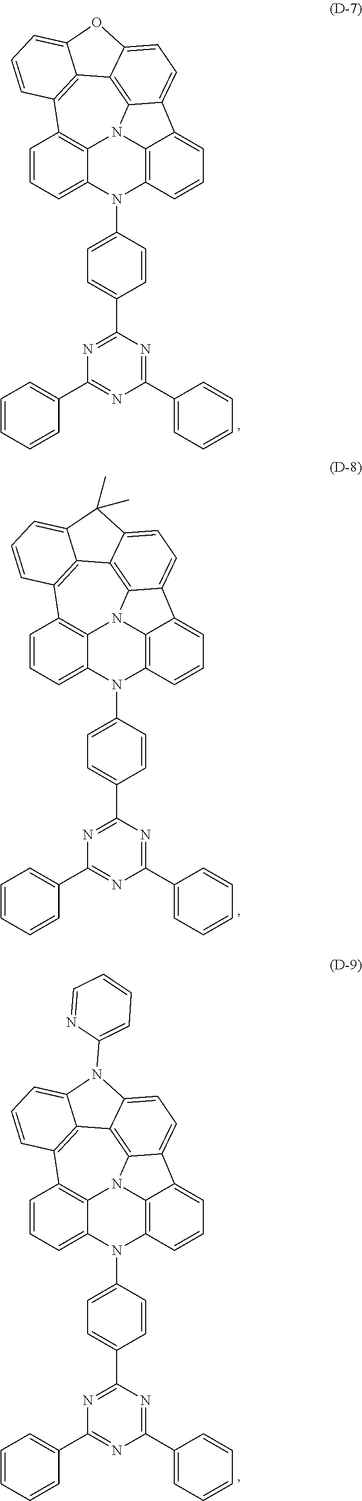 [SMILES: c1ccc(-c2nc(-c3ccccc3)nc(-c3ccc(N4c5cccc6c7cccc8oc9ccc%10c%11cccc4c%11n(c56)c%10c9c87)cc3)n2)cc1 ; CC1(C)c2cccc3c2-c2c1ccc1c4cccc5c4n(c21)-c1c-3cccc1N5c1ccc(-c2nc(-c3ccccc3)nc(-c3ccccc3)n2)cc1 ; c1ccc(-c2nc(-c3ccccc3)nc(-c3ccc(N4c5cccc6c7cccc8c7c7c(ccc9c%10cccc4c%10n(c56)c97)n8-c4ccccn4)cc3)n2)cc1]

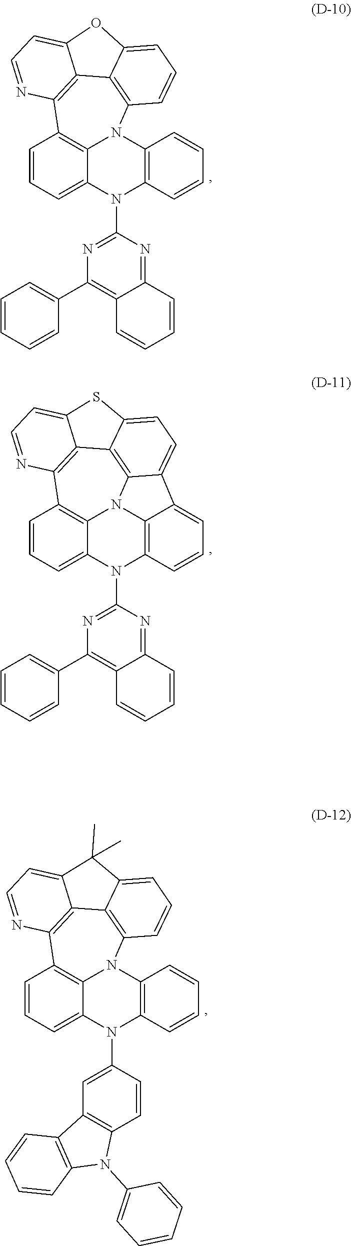 [SMILES: c1ccc(-c2nc(N3c4ccccc4-n4c5cccc6oc7ccnc(c8cccc3c84)c7c65)nc3ccccc23)cc1 ; c1ccc(-c2nc(N3c4cccc5c6ccc7sc8ccnc9c%10cccc3c%10n(c45)c6c7c89)nc3ccccc23)cc1 ; CC1(C)c2cccc3c2-c2c1ccnc2-c1cccc2c1N3c1ccccc1N2c1ccc2c(c1)c1ccccc1n2-c1ccccc1]

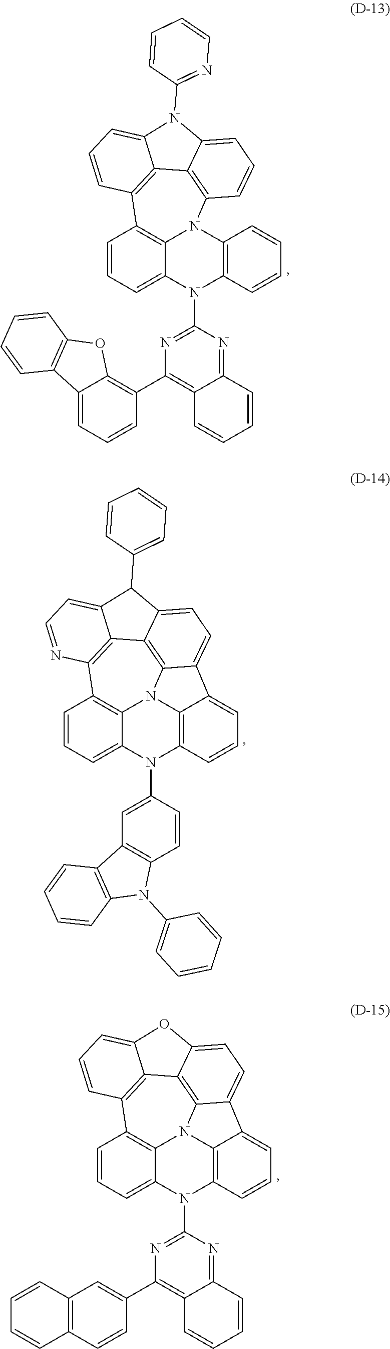 [SMILES: c1ccc(-n2c3cccc4c5cccc6c5n(c5cccc2c5c43)-c2ccccc2N6c2nc(-c3cccc4c3oc3ccccc34)c3ccccc3n2)nc1 ; c1ccc(C2c3ccnc4c3-c3c2ccc2c5cccc6c5n(c32)-c2c-4cccc2N6c2ccc3c(c2)c2ccccc2n3-c2ccccc2)cc1 ; c1ccc2cc(-c3nc(N4c5cccc6c7cccc8oc9ccc%10c%11cccc4c%11n(c56)c%10c9c87)nc4ccccc34)ccc2c1]

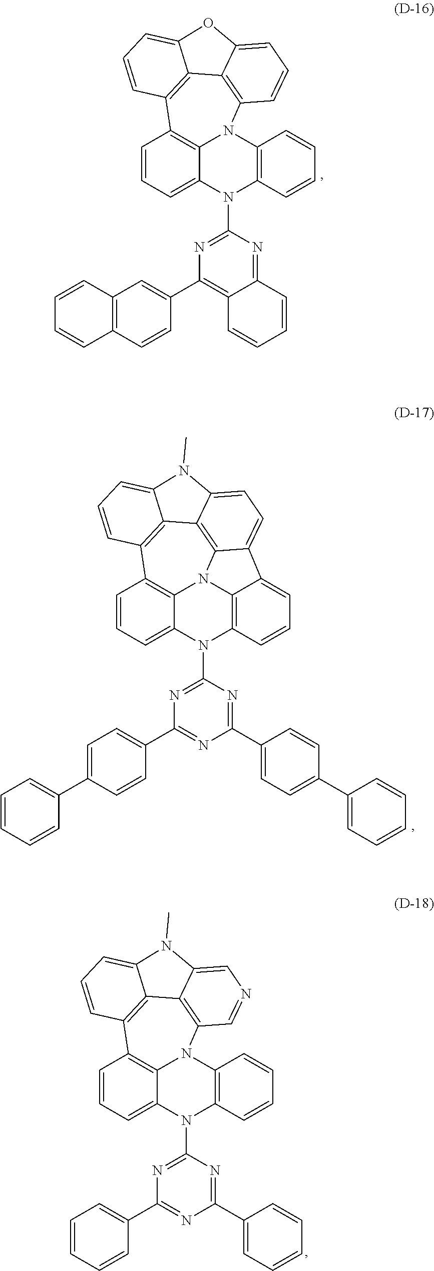 [SMILES: c1ccc2c(c1)N(c1nc(-c3ccc4ccccc4c3)c3ccccc3n1)c1cccc3c4cccc5oc6cccc(c6c54)n-2c13 ; Cn1c2cccc3c4cccc5c4n4c6c(cccc6c6ccc1c(c32)c64)N5c1nc(-c2ccc(-c3ccccc3)cc2)nc(-c2ccc(-c3ccccc3)cc2)n1 ; Cn1c2cccc3c4cccc5c4n(c4cncc1c4c32)-c1ccccc1N5c1nc(-c2ccccc2)nc(-c2ccccc2)n1]

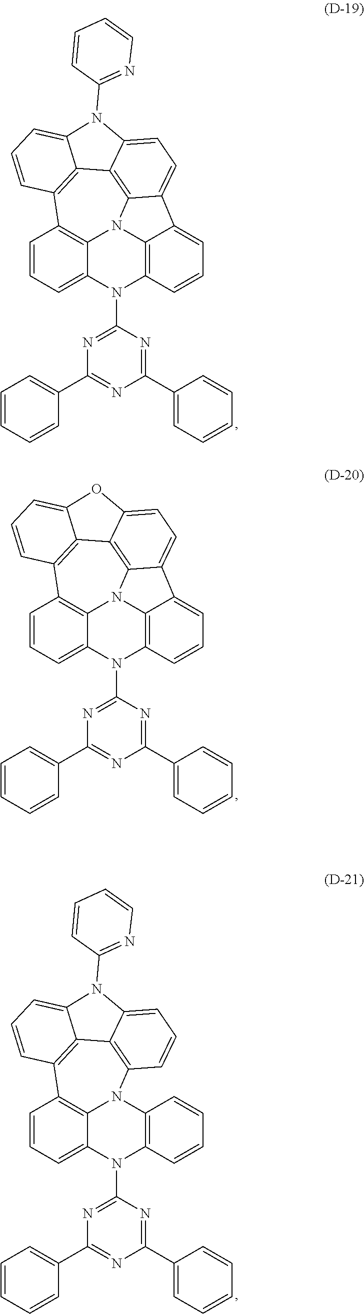 [SMILES: c1ccc(-c2nc(-c3ccccc3)nc(N3c4cccc5c6cccc7c6c6c(ccc8c9cccc3c9n(c45)c86)n7-c3ccccn3)n2)cc1 ; c1ccc(-c2nc(-c3ccccc3)nc(N3c4cccc5c6cccc7oc8ccc9c%10cccc3c%10n(c45)c9c8c76)n2)cc1 ; c1ccc(-c2nc(-c3ccccc3)nc(N3c4ccccc4-n4c5cccc6c5c5c(cccc5n6-c5ccccn5)c5cccc3c54)n2)cc1]

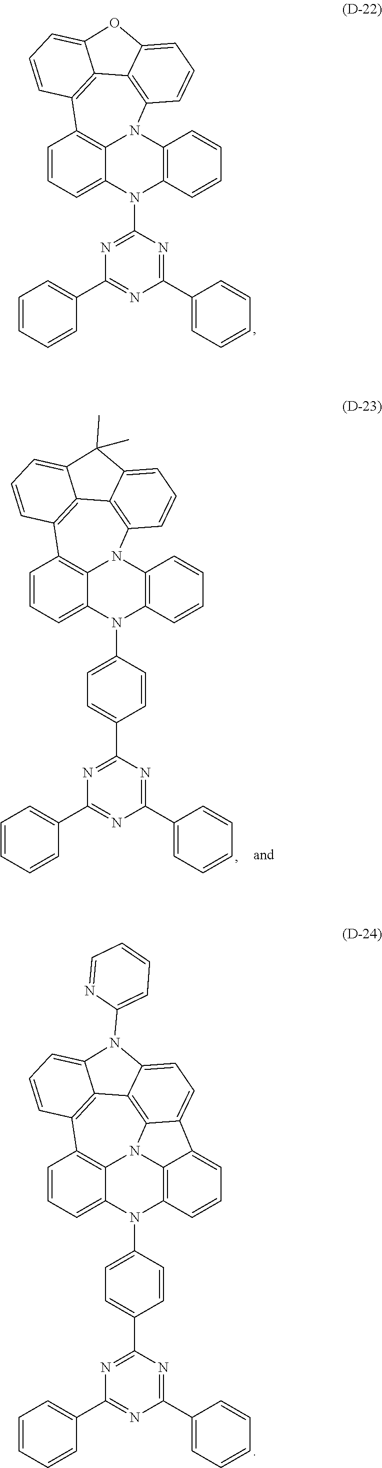 [SMILES: c1ccc(-c2nc(-c3ccccc3)nc(N3c4ccccc4-n4c5cccc6oc7cccc(c8cccc3c84)c7c65)n2)cc1 ; CC1(C)c2cccc3c2-c2c(cccc21)N1c2ccccc2N(c2ccc(-c4nc(-c5ccccc5)nc(-c5ccccc5)n4)cc2)c2cccc-3c21 ; c1ccc(-c2nc(-c3ccccc3)nc(-c3ccc(N4c5cccc6c7cccc8c7c7c(ccc9c%10cccc4c%10n(c56)c97)n8-c4ccccn4)cc3)n2)cc1]

In a second aspect, the present invention provides a method for preparing the fused cyclic compound.

The compound of Formula (I) is synthesized through the following steps:

subjecting a compound of Formula (A) and a compound of Formula (B) as starting materials to a coupling reaction in the presence of a catalyst, to obtain an intermediate 1; cyclizing the intermediate 1, to obtain an intermediate 2; and subjecting the intermediate 2 and a compound T₃-L-Ar₁ to a substitution or coupling reaction in the presence of a catalyst, to obtain the compound of Formula (I).

The synthesis route for the compound of Formula (I) is shown below:

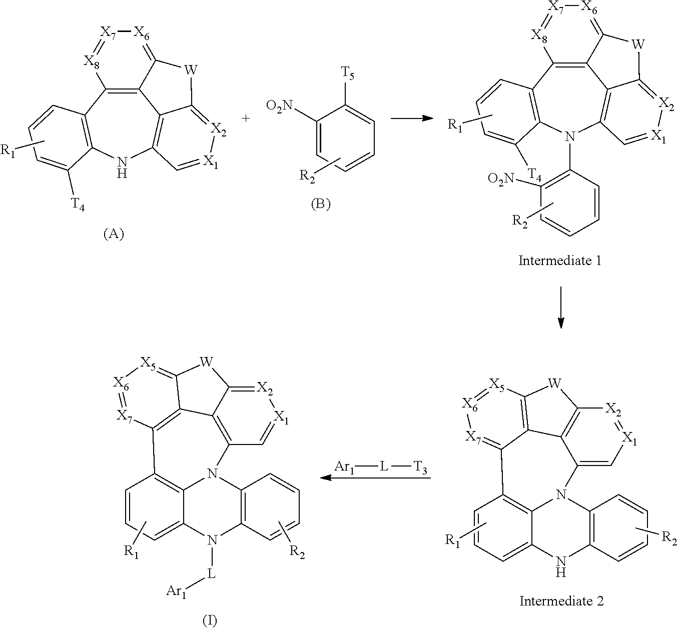

The compound of Formula (II) is synthesized through the following steps:

subjecting a compound of Formula (C) and a compound of Formula (E) as starting materials to a coupling reaction in the presence of a catalyst, to obtain an intermediate 3; cyclizing the intermediate 3, to obtain an intermediate 4; reducing the nitro group of the intermediate 4 and then subjecting the intermediate 4 to a coupling reaction, to obtain an intermediate 5; and subjecting the intermediate 5 and a compound T₃-L-Ar₁ to a substitution or coupling reaction in the presence of a catalyst, to obtain the compound of Formula (II).

The synthesis route for the compound of Formula (II) is shown below:

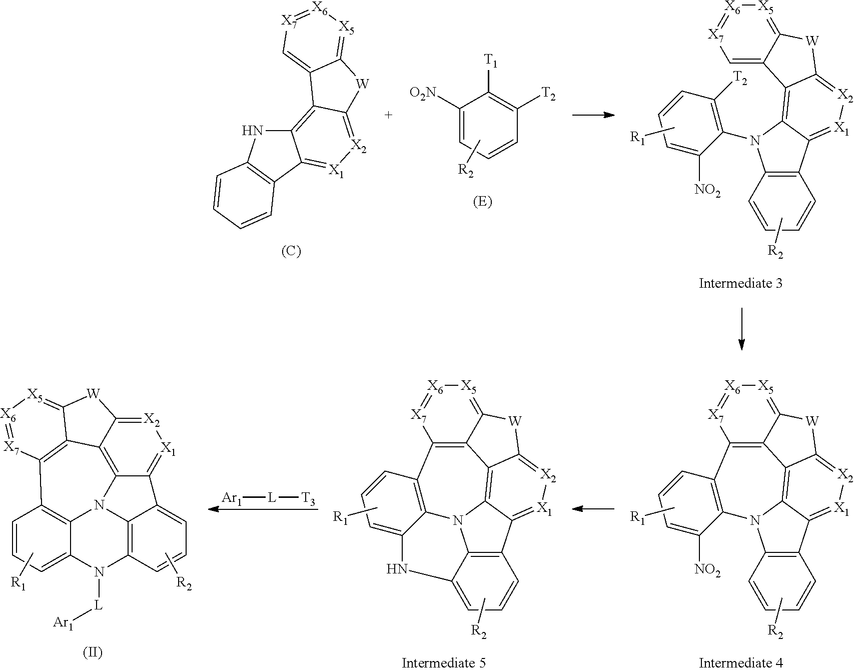

where T₁-T₅ are each independently selected from hydrogen, fluoro, chloro, bromo or iodo.

In a third aspect, the present invention provides use of the fused cyclic compound as an organic electroluminescent material.

In a fourth aspect, the present invention provides an organic light-emitting device, having at least one functional layer containing the fused cyclic compound.

Preferably, in the organic light-emitting device, the functional layer is a light-emitting layer.

Further preferably, the material of the light-emitting layer in the organic light-emitting device comprises a host material and a guest luminescent dye, where the host material is the fused cyclic compound.

The technical solution of the invention has the following advantages.

1. The fused cyclic compound provided in present invention has a structure of Formula (I) or (II). The fused cyclic compound has increased effective conjugation of the mother nuclear structure by designing the fusion mode of the aromatic ring and the heterocyclic ring in the mother nuclear structure, and facilitates the balancing of the electron transport performance of material molecules while improving the hole performance of the fused cyclic compound. By controlling the degree of conjugation of the molecules, the HOMO level of the fused cyclic compound is increased, and the difference between the singlet energy level and the triplet energy level of the molecule of material is reduced. When the material is used as a host material in the light-emitting layer, the HOMO level of the light-emitting layer can be made more matched with that of the hole injection layer, thus facilitating the injection of holes.

By configuring X₁-X₇, the fused cyclic compound is allowed to have both electron transport performance and hole transport performance. When the fused cyclic compound is used as a host material in the light-emitting layer, the ratio of electrons and holes in the light-emitting layer can be balanced, the carrier recombination probability is improved, and the carrier recombination region is widened, thereby improving the luminescence efficiency.

On the other hand, the fused cyclic compound of Formula (I) or Formula (II) has a high triplet energy level (T₁) and a high glass transition temperature, and can promote the effective energy transfer from the host material to the guest material, reduce the returning of the energy and improve the luminescence efficiency of OLED devices, due to the high triplet energy level when used as a host material in the light emitting layer. The fused cyclic compound has a high glass transition temperature, high thermal stability and morphological stability, and excellent film forming performance, and is not prone to crystallization when used as a host material in the light emitting layer, thus facilitating the improvement of the performance and luminescence efficiency of the OLED device.

2. In the fused cyclic compound provided in the invention, by adjusting the substituents R₁, R₂, R₁ₐ-R₇ₐ, Ar₁, and Ar₂, an electron withdrawing group (pyridine, pyrimidine, triazine, pyrazine, oxadiazole, thiadiazole, quinazoline, imidazole, quinoxaline, quinoline, and the like), or an electron donating group (diphenylamine, triphenylamine, fluorene, and the like) can be introduced on the substituents, where the HOMO level is distributed on the electron donating group, and the LUMO level is distributed on the electron withdrawing group, such that the hole transport performance and the electron transport performance of the material molecules are further improved, and the balance of charge transfer is improved. When the material is used as a host material in the light emitting layer, the recombination region of holes and electrons is further enlarged, and the exciton concentration per unit volume is diluted to prevent concentration quenching or triplet-triplet exciton annihilation of triplet excitons due to high concentration. By configuring the electron donating group and the electron withdrawing group, the HOMO level of the fused cyclic compound is improved, and the LUMO level is lowered. When the material is used as a host material in the light emitting layer, the adjacent hole- and electron-type carrier functional layers are allowed to become more matched.

In the fused cyclic compound as shown in FIG. 1 (the compound shown in FIG. 1 is the fused cyclic compound D-3), the HOMO level and LUMO level are distributed on different electron donating group and electron withdrawing group, such that the HOMO level is effectively separated from the LUMO level. This reduces the difference ΔEst (≤0.3 eV) between the singlet and triplet energy levels of the material molecule, thus facilitating the reverse intersystem crossing of the triplet excitons to singlet excitons, promoting the FÖrster energy transfer from the host material to the guest material, and reducing losses generated during energy transfer.

By configuring the electron-donating group, the electron withdrawing group and their spatial positions, a twisted rigid molecular configuration is attained, and the degree of intermolecular conjugation is adjusted to further increase the triplet energy level of the material molecule, so a small ΔEst is achieved. Moreover, by configuring L and Ar₁, and adjusting the electron-donating group, the electron withdrawing group, and the spacing therebetween, the LUMO level or the HOMO level is allowed to distribute more evenly, thus further optimizing the HOMO and LUMO levels.

3. The preparation method of the fused cyclic compound provided in the present invention has the advantages of readily available starting materials, mild reaction conditions, and simple operation steps, so a simple and easy-to-implement preparation method is provided for the mass production of the above fused cyclic compound.

4. The organic light-emitting diode (OLED) device provided in the present invention has at least one functional layer containing the fused cyclic compound, where the functional layer is a light emitting layer.

The fused cyclic compound makes the electron and hole transport performances balanced, and increases the recombination probability of electrons and holes in the light emitting layer. Moreover, the fused cyclic compound has a high triplet energy level, which facilitates the energy transfer of the host material to the guest material and prevents the returning of energy. The high glass transition temperature of the fused cyclic compound prevents crystallization of the material molecules of the light emitting layer and improves the performance of the OLED device during use.

By adjusting the substituents, the electron and hole transport performances of the fused cyclic compound are further improved, and the transport of charges and holes in the light emitting layer becomes more balanced, thereby expanding the area where holes and electrons are recombined into excitons in the light emitting layer, reducing the exciton concentration, preventing the triplet-triplet annihilation of the device, and improving the efficiency of the device; and also the carrier recombination region is allowed to stay far away from the interface of the light emitting layer to the hole or electron transport layer, thus improving the color purity of the OLED device, avoiding the returning of excitons to the transport layer, and further improving the efficiency of the device.

In the fused cyclic compound, an electron donating group and an electron withdrawing group are utilized to adjust the HOMO level and the LUMO level of the material molecules, so that the overlap between the HOMO level and the LUMO level is reduced and the fused ring has a small ΔEst, which promotes the reverse intersystem crossing (RISC) of triplet excitons to singlet excitons, thereby suppressing the Dexter energy transfer (DET) from the host material to the luminescent dye, promoting the FÖrster energy transfer, and reducing the energy loss during the Dexter energy transfer (DET). In this way, the efficiency roll-off of the organic light-emitting device is effectively reduced, and the external quantum efficiency of the device is improved.

### BRIEF DESCRIPTION OF THE DRAWINGS

In order to more clearly explain the technical solutions in the specific embodiments of the present invention or in the prior art, the drawings used in the description of the specific embodiments or the prior art will be briefly described below. Obviously, the drawings depicted below are merely some embodiments of the present invention, and those skilled in the art can obtain other drawings based on these drawings without any creative efforts.

FIG. 1 is a view showing the theoretical calculation results of HOMO level, LOMO level and ΔEst of the fused cyclic compound D-3 prepared in Example 1 of the present invention; and

FIG. 2 is a schematic structural view of an organic light-emitting device according to Examples 8 to 14 and Comparative Example 1 of the present invention.

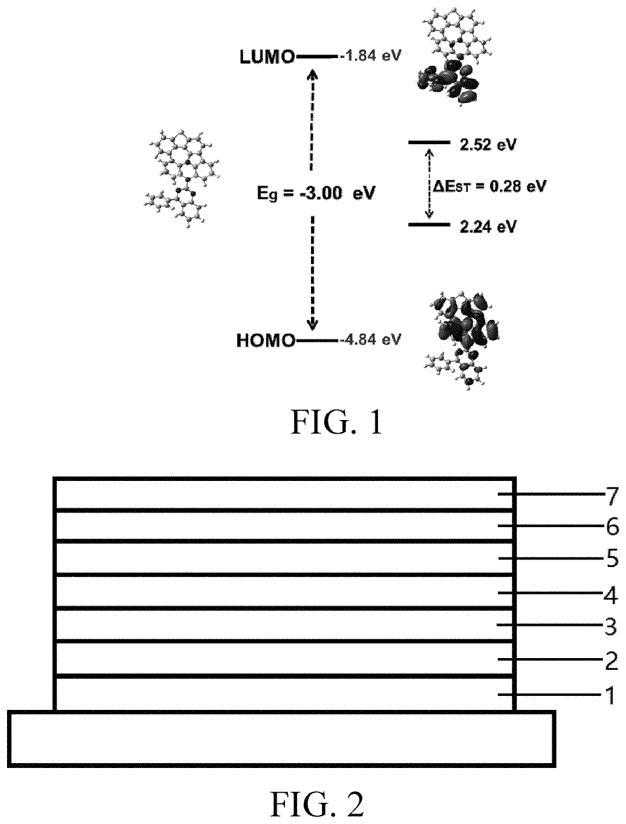

### LIST OF REFERENCE NUMERALS

1—anode, 2—hole injection layer, 3—hole transport layer, 4—light emitting layer, 5—electron transport layer, 6—electron injection layer, 7—cathode.

### DETAILED DESCRIPTION

The technical solutions of the present invention will be described clearly and fully with reference to the accompanying drawings. Apparently, the embodiments described are some preferred embodiments, rather than all of the embodiments of the present invention. All other embodiments obtained by a person of ordinary skill in the art without creative efforts based on the embodiments of the present invention shall fall within the protection scope of the present invention.

It is to be understood that in the description of the present invention, the terms “first” and “second” are used herein for purposes of description, and are not intended to indicate or imply relative importance.

The present invention can be embodied in many different forms and is not limited to the embodiments described herein. Conversely, these embodiments are provided for the purpose of making the disclosure of the present invention more thorough and comprehensive and conveying the concept of the present invention fully to those skilled in the art, and the scope of the present invention is defined merely by the claims. In the figures, for the sake of clarity, the dimensions and relative dimensions of the layers and regions will be exaggerated. It should be understood that when an element, for example, a layer, is referred to as being “formed” or “disposed” “on” another element, the element may be directly disposed on the other element or an intervening element may be present. Conversely, when an element is referred to as being “directly formed on” or “directly disposed on” another element, no intervening element is present.

### Example 1

This example provides a fused cyclic compound having a structure of Formula D-3 below:

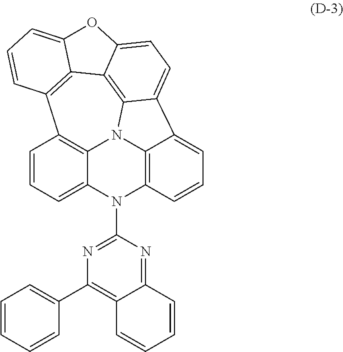 [SMILES: c1ccc(-c2nc(N3c4cccc5c6cccc7oc8ccc9c%10cccc3c%10n(c45)c9c8c76)nc3ccccc23)cc1]

The synthesis route for the fused cyclic compound of Formula D-3 is shown below:

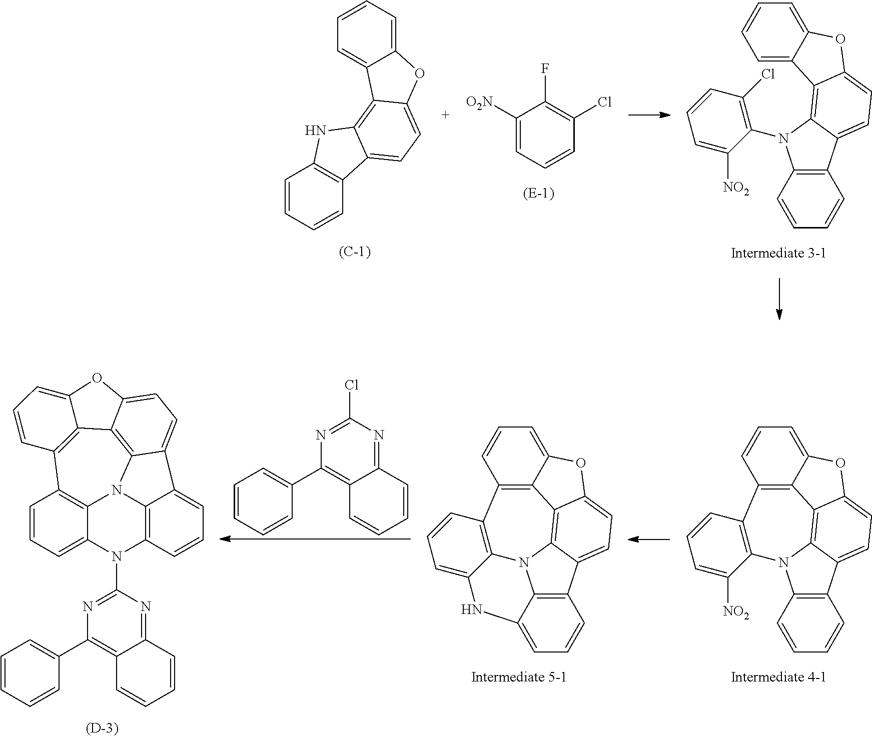

The method for preparing the fused cyclic compound of Formula D-3 comprises specifically the following steps.

(1) Synthesis of Intermediate 3-1

Under nitrogen atmosphere, a compound of Formula (C-1) (12.8 g, 50 mmol), 3-chloro-2-fluoronitrobenzene (8.8 g, 50 mmol) (a compound of Formula (E-1)), cesium carbonate (19.5 g, 60 mmol), and dimethyl sulfoxide (200 mL) were added to a 500 mL three-neck flask, and reacted for 15 hrs. The reaction solution was extracted with toluene, and then the solvent was removed by rotary evaporation. The residue was purified by column chromatography on silica gel, to obtain the intermediate 3-1 as a solid (14.8 g, yield 72%).

(2) Synthesis of Intermediate 4-1

Under nitrogen atmosphere, the intermediate 3-1 (12.4 g, 30 mmol), palladium diacetate (0.6 g, 3.0 mmol), tricyclohexylphosphonium tetrafluoroborate (2.2 g, 6.0 mmol), cesium carbonate (29.1 g, 90 mmol), and o-xylene (150 mL) were added to a 500 mL three-neck flask, and reacted under reflux with heating for 2 hrs. The reaction solution was extracted with chloroform, and then the solvent was removed by rotary evaporation. The residue was purified by column chromatography on silica gel, to obtain the intermediate 4-1 as a solid (8.5 g, yield 75%).

(3) Synthesis of Intermediate 5-1

Under nitrogen atmosphere, the intermediate 4-1 (7.9 g, 21 mmol), stannous chloride dihydrate (18.9 g, 84 mmol), hydrochloric acid (15 mL), and ethanol (120 mL) were added, and reacted for 10 hrs at 60° C. The reaction solution was extracted with chloroform, washed with water and then with brine, and dried over anhydrous magnesium sulfate, and then the solvent was removed by rotary evaporation. After drying, the residue was transferred to a reaction flask, and tris(dibenzylideneacetone)dipalladium (0.19 g, 0.21 mmol) and toluene (150 mL) were added, reacted for 8 hrs at 110° C., and then cooled to room temperature. The reaction solution was extracted with chloroform and washed with water, and then the solvent was removed by rotary evaporation. The residue was purified by column chromatography on silica gel, to obtain the intermediate 5-1 as a solid (5.13 g, yield 71%).

(4) Synthesis of Fused Cyclic Compound D-3

Under nitrogen atmosphere, the intermediate 5-1 (3.4 g, 10 mmol), the compound

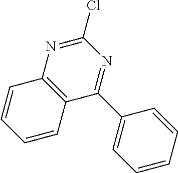 [SMILES: Clc1nc(-c2ccccc2)c2ccccc2n1]

(3.2 g, 12 mmol), cesium carbonate (3.4 g, 10 mmol), 4-dimethylamino pyridine (0.6 g, 5.0 mmol), and dimethyl sulfoxide (40 mL) were added, reacted at 100° C. for 3 hrs, and then cooled to room temperature. The reaction solution was extracted with toluene, and then the solvent was removed by rotary evaporation. The residue was purified by column chromatography on silica gel, to obtain the compound D-3 as a solid (4.6 g, yield 85%).

Element analysis: (C38H20N4O) calculated: C, 83.20; H, 3.67; N, 10.21; O, 2.92. found: C, 83.16; H, 3.69; N, 10.19; O, 2.96. HRMS (ESI) m/z (M+): calculated: 548.16. found: 548.32.

### Example 2

This example provides a fused cyclic compound having a structure of Formula D-4 below:

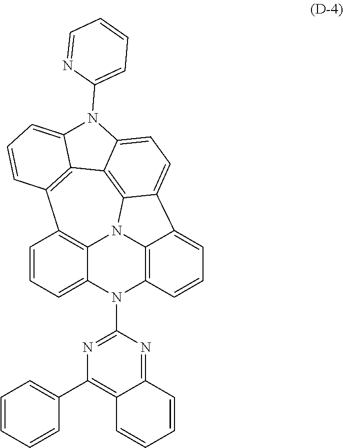 [SMILES: c1ccc(-c2nc(N3c4cccc5c6cccc7c6c6c(ccc8c9cccc3c9n(c45)c86)n7-c3ccccn3)nc3ccccc23)cc1]

The synthesis route for the fused cyclic compound of Formula D-4 is shown below:

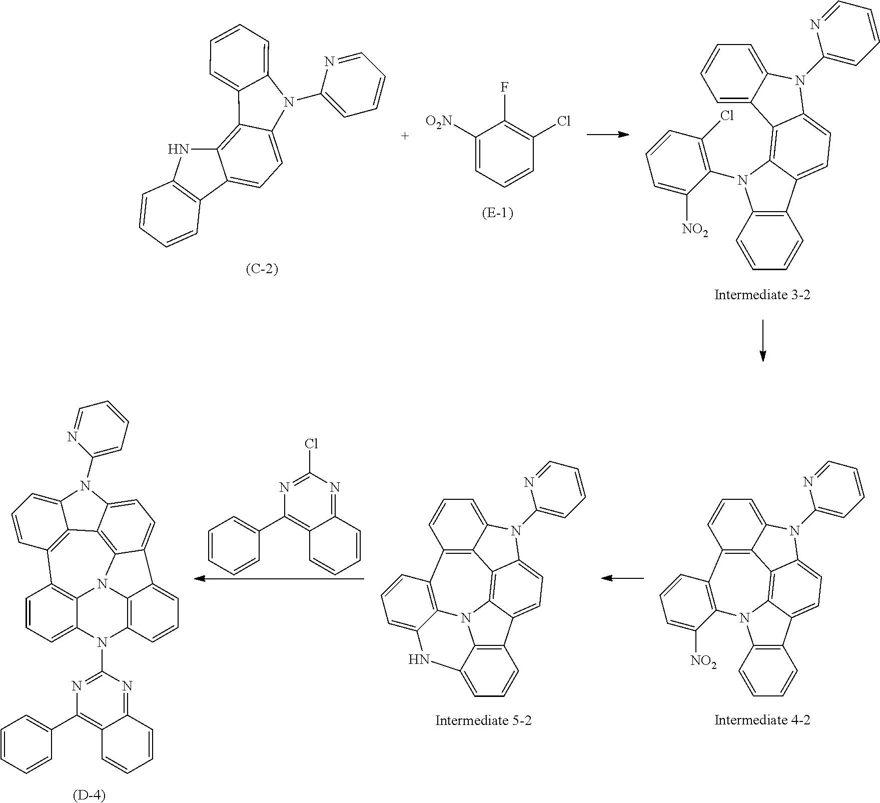

The method for preparing the fused cyclic compound of Formula D-4 comprises specifically the following steps.

Starting with the compound of Formula (C-2) and the compound of Formula (E-1), a fused cyclic compound of Formula (D-4) was obtained following the synthesis method in Example 1.

Element analysis: (C43H24N6) calculated: C, 82.67; H, 3.87; N, 13.45. found: C, 82.61; H, 3.83; N, 13.49. HRMS (ESI) m/z (M+): calculated: 624.21. found: 624.19.

### Example 3

This example provides a fused cyclic compound having a structure of Formula D-1 below:

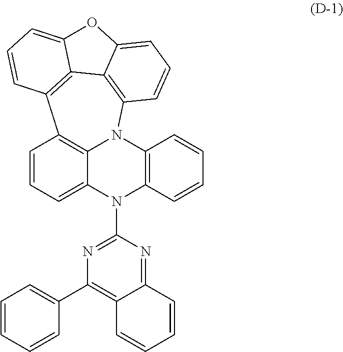 [SMILES: c1ccc(-c2nc(N3c4ccccc4-n4c5cccc6oc7cccc(c8cccc3c84)c7c65)nc3ccccc23)cc1]

The synthesis route for the fused cyclic compound of Formula D-1 is shown below:

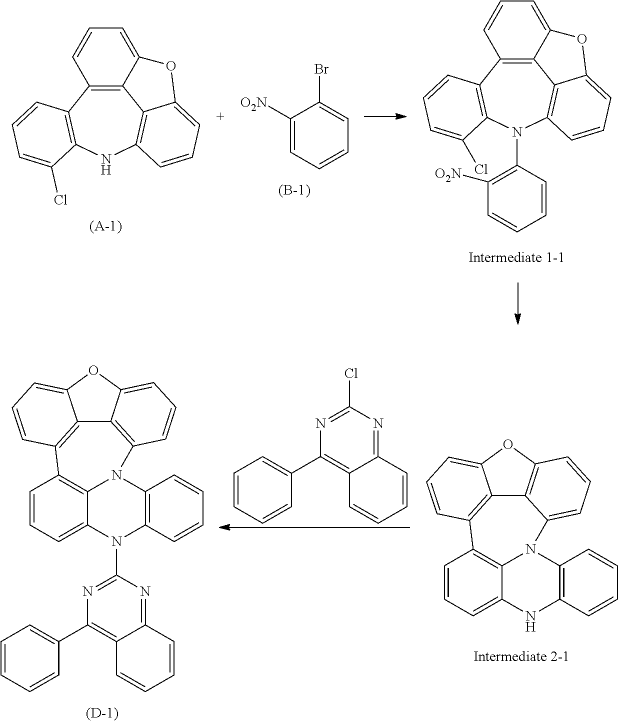

(1) Synthesis of Intermediate 1-1

Under nitrogen atmosphere, a compound of Formula (A-1) (8.7 g, 30 mmol), 1-bromo-2-nitrobenzene (6.7 g, 33 mmol), palladium diacetate (0.2 g, 1.0 mmol), tri-tert-butylphosphine (0.66 g, 3.5 mmol), sodium-t-butoxide (9.3 g), and toluene (1000 mL) were weighed, reacted at 110° C. for 12 hrs, and then cooled to room temperature. The reaction solution was extracted with chloroform, and then the solvent was removed by rotary evaporation. The residue was purified by column chromatography on silica gel, to obtain the intermediate 1-1 as a solid (10.5 g, yield 85%).

(2) Synthesis of Intermediate 2-1

Under nitrogen atmosphere, the intermediate 1-1 (8.2 g, 20 mmol), stannous chloride dihydrate (21.6 g, 96 mmol), hydrochloric acid (17 mL), and ethanol (250 mL) were added, and reacted for 10 hrs at 60° C. The reaction solution was extracted with chloroform, washed with water and then with brine, and dried over anhydrous magnesium sulfate, and then the solvent was removed by rotary evaporation. After drying, the residue was transferred to a reaction flask, and tris(dibenzylideneacetone)dipalladium (0.22 g, 0.24 mmol) and toluene (200 mL) were added, reacted for 8 hrs at 110° C., and then cooled to room temperature. The reaction solution was extracted with chloroform and washed with water, and then the solvent was removed by rotary evaporation. The residue was purified by column chromatography on silica gel, to obtain the intermediate 2-1 as a solid (4.8 g, yield 70%).

(3) Synthesis of Fused Cyclic Compound D-1

Under nitrogen atmosphere, the intermediate 2-1 (3.5 g, 10 mmol), the compound

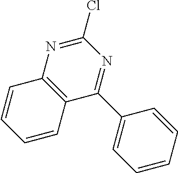 [SMILES: Clc1nc(-c2ccccc2)c2ccccc2n1]

(3.2 g, 12 mmol), cesium carbonate (3.4 g, 10 mmol), 4-dimethylamino pyridine (0.6 g, 5 mmol), and dimethyl sulfoxide (40 mL) were added, reacted at 100° C. for 3 hrs, and then cooled to room temperature. The reaction solution was extracted with toluene, and then the solvent was removed by rotary evaporation. The residue was purified by column chromatography on silica gel, to obtain the fused cyclic compound D-1 as a solid (4.7 g, yield 85%).

Element analysis: (C38H22N4O) calculated: C, 82.89; H, 4.03; N, 10.18; O, 2.91. found: C, 82.83; H, 4.08; N, 10.21; O, 2.84. HRMS (ESI) m/z (M+): calculated: 550.18. found: 550.31.

### Example 4

This example provides a fused cyclic compound having a structure of Formula D-5 below:

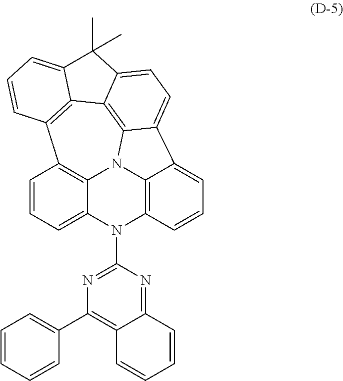 [SMILES: CC1(C)c2cccc3c2-c2c1ccc1c4cccc5c4n(c21)-c1c-3cccc1N5c1nc(-c2ccccc2)c2ccccc2n1]

The synthesis route for the fused cyclic compound of Formula D-5 is shown below:

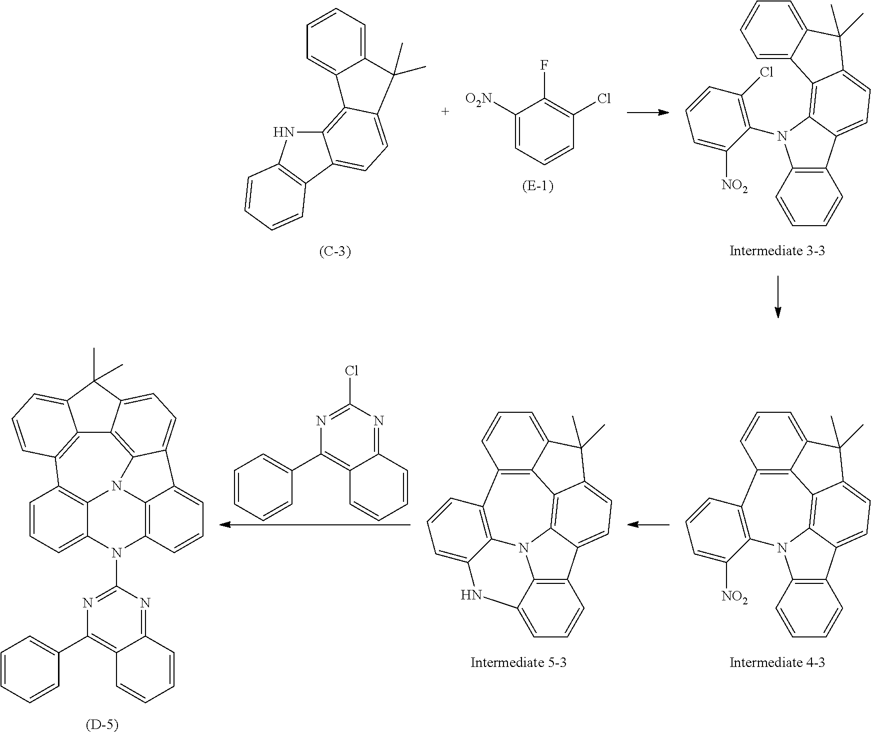

The method for preparing the fused cyclic compound of Formula D-5 comprises specifically the following steps.

Starting with the compound of Formula (C-3) and the compound of Formula (E-1), a fused cyclic compound of Formula (D-5) was obtained following the synthesis method in Example 1.

Element analysis: (C41H26N4) calculated: C, 85.69; H, 4.56; N, 9.75. found: C, 85.67; H, 4.57; N, 9.78. HRMS (ESI) m/z (M+): calculated: 574.22. found: 574.34.

### Example 5

This example provides a fused cyclic compound having a structure of Formula D-6 below:

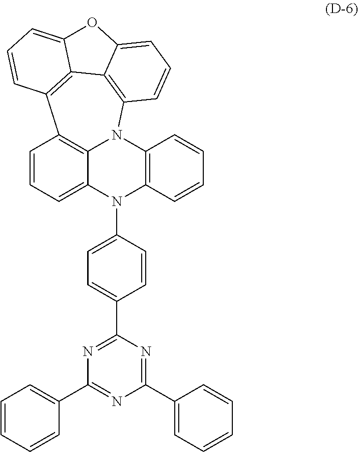 [SMILES: c1ccc(-c2nc(-c3ccccc3)nc(-c3ccc(N4c5ccccc5-n5c6cccc7oc8cccc(c9cccc4c95)c8c76)cc3)n2)cc1]

The synthesis route for the fused cyclic compound of Formula D-6 is shown below:

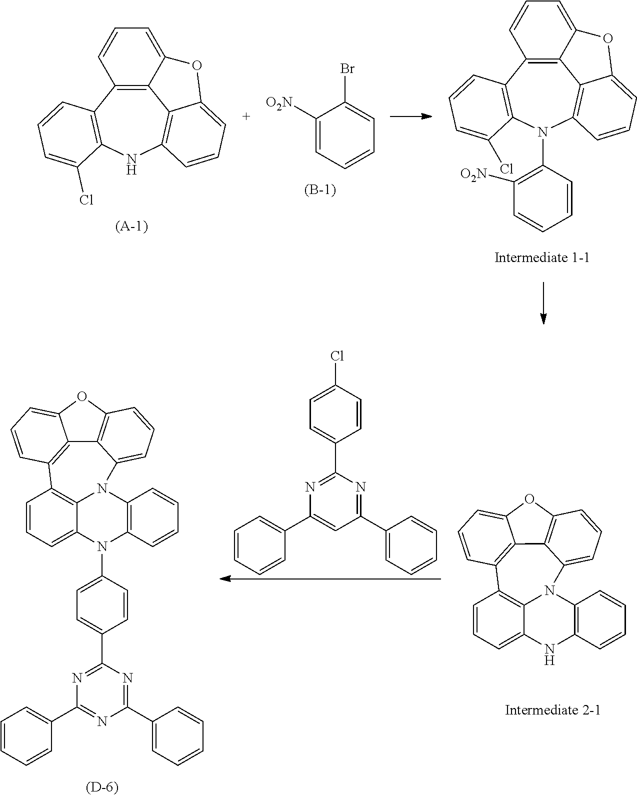

The method for preparing the fused cyclic compound of Formula D-6 comprises specifically the following steps.

(1) The intermediate 2-1 was synthesized following the method as described in Example 3.

(2) Synthesis of Fused Cyclic Compound D-6

Under nitrogen atmosphere, Intermediate 2-1 (3.4 g, 10 mmol), palladium diacetate (0.06 g, 0.3 mmol), tri-tert-butylphosphine (0.2 g, 1.1 mmol), the compound

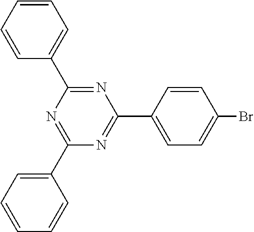 [SMILES: Brc1ccc(-c2nc(-c3ccccc3)nc(-c3ccccc3)n2)cc1]

(4 g, 10.2 mmol), sodium-t-butoxide (2.8 g), and 1000 mL (toluene) were added, reacted at 110° C. for 12 hrs, and then cooled to room temperature. The reaction solution was extracted with chloroform, and then the solvent was removed by rotary evaporation. The residue was purified by column chromatography on silica gel, to obtain the fused cyclic compound D-6 as a solid (5.3 g, yield 82%).

Element analysis: (C45H25N5O) calculated: C, 82.93; H, 3.87; N, 10.75; O, 2.45. found: C, 82.90; H, 3.89; N, 10.71; O, 2.47. HRMS (ESI) m/z (M+): calculated: 651.21. found: 651.27.

### Example 6

This example provides a fused cyclic compound having a structure of Formula D-9 below:

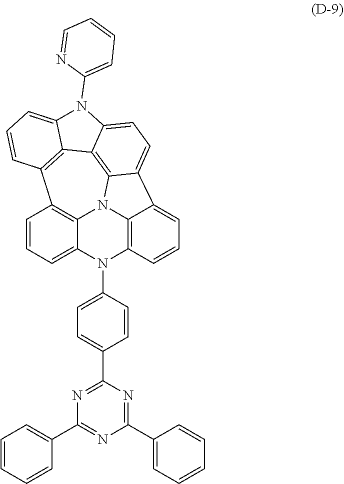 [SMILES: c1ccc(-c2nc(-c3ccccc3)nc(-c3ccc(N4c5cccc6c7cccc8c7c7c(ccc9c%10cccc4c%10n(c56)c97)n8-c4ccccn4)cc3)n2)cc1]

The synthesis route for the fused cyclic compound of Formula D-9 is shown below:

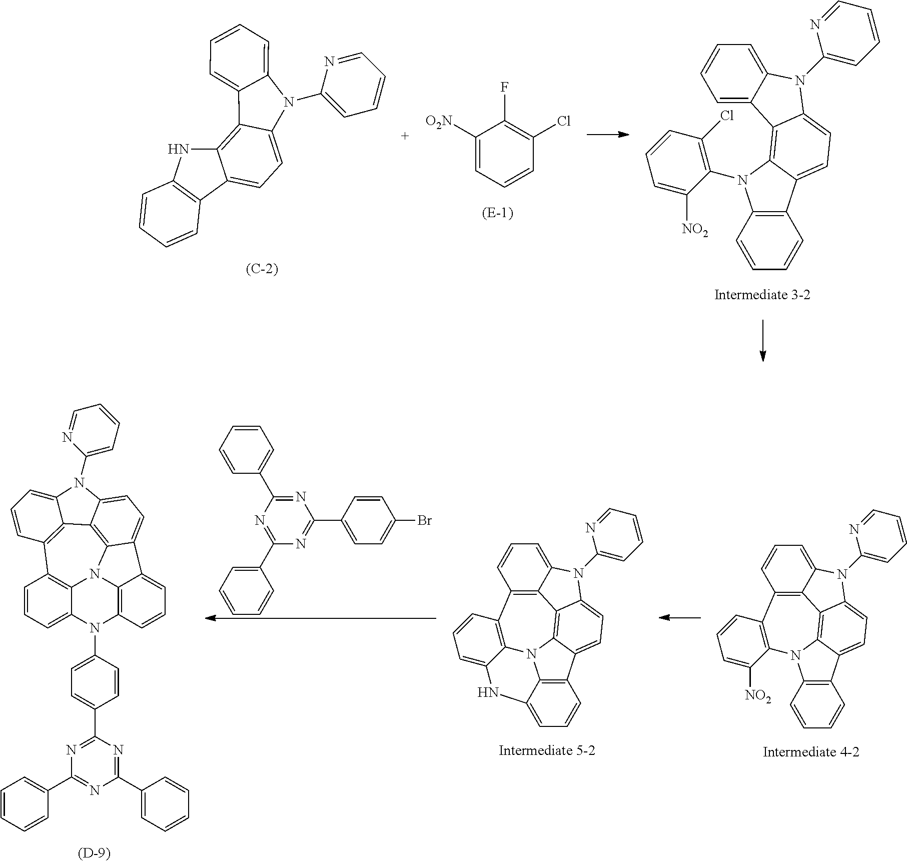

The method for preparing the fused cyclic compound of Formula D-4 comprises specifically the following steps.

Starting with the compound of Formula (C-2) and the compound of Formula (E-1), a fused cyclic compound of Formula (D-9) was obtained following the synthesis method in Example 5.

Element analysis: (C50H29N7) calculated: C, 82.51; H, 4.02; N, 13.47. found: C, 82.47; H, 4.07; N, 13.42. HRMS (ESI) m/z (M+): calculated: 727.25. found: 727.31.

### Example 7

This example provides a fused cyclic compound having a structure of Formula D-8 below:

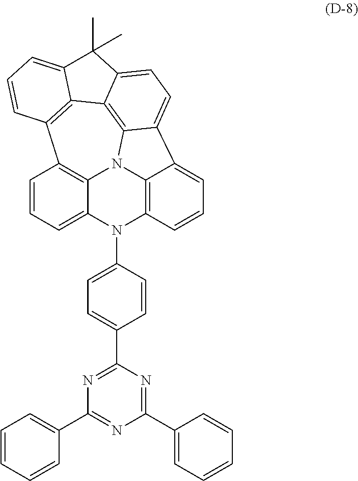 [SMILES: CC1(C)c2cccc3c2-c2c1ccc1c4cccc5c4n(c21)-c1c-3cccc1N5c1ccc(-c2nc(-c3ccccc3)nc(-c3ccccc3)n2)cc1]

The synthesis route for the fused cyclic compound of Formula D-8 is shown below:

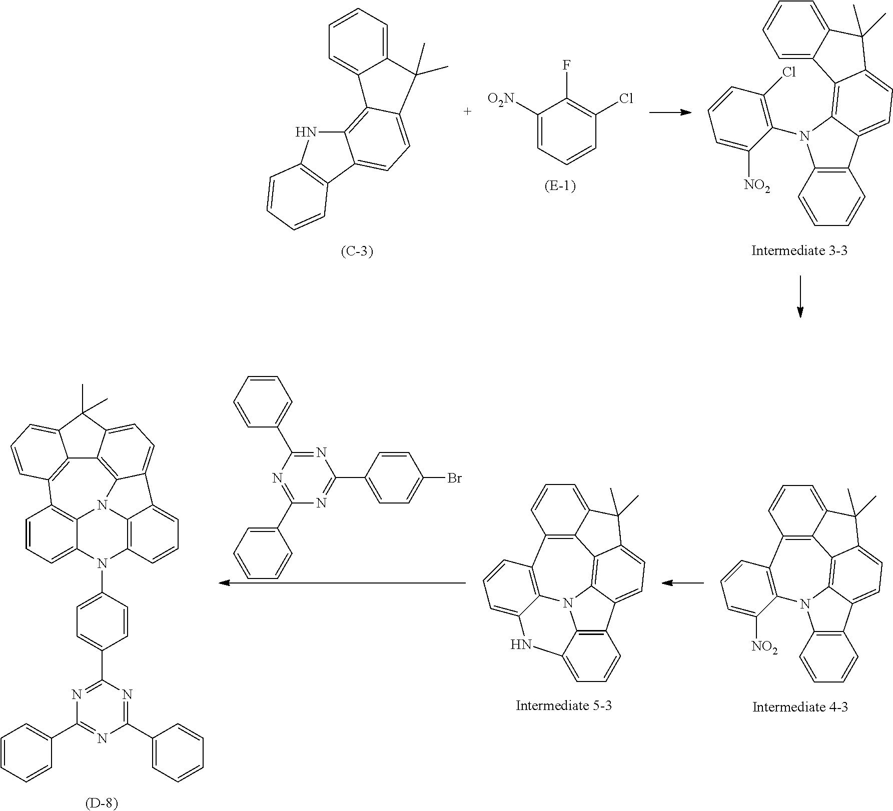

The method for preparing the fused cyclic compound of Formula D-8 comprises specifically the following steps.

Starting with the compound of Formula (C-3) and the compound of Formula (E-1), a fused cyclic compound of Formula (D-8) was obtained following the synthesis method in Example 5.

Element analysis: (C48H31N5) calculated: C, 85.06; H, 4.61; N, 10.33. found: C, 85.01; H, 4.67; N, 10.34. HRMS (ESI) m/z (M+): calculated: 677.26. found: 677.34.

### Example 8

This example provides an organic light-emitting device, which includes, from bottom to top, an anode 2, a hole injection layer 1, a hole transport layer 3, a light emitting layer 4, an electron transport layer 5, an electron injection layer 6 and a cathode 7 stacked in sequence. as shown in FIG. 2.

In the organic light-emitting device, the material of the anode is ITO; and the material of the cathode 7 is the metal Al.

The material of the hole injection layer 2 is HAT(CN)6, having a chemical structure as shown below:

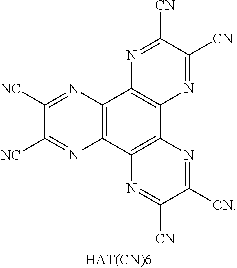

The material of the hole transport layer 3 is a compound having a structure below:

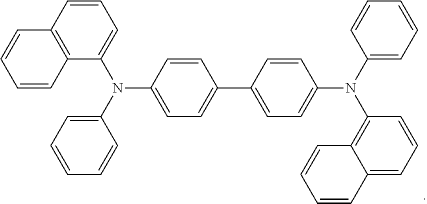 [SMILES: c1ccc(N(c2ccc(-c3ccc(N(c4ccccc4)c4cccc5ccccc45)cc3)cc2)c2cccc3ccccc23)cc1]

The material of the electron transport layer 5 is a compound having a structure below:

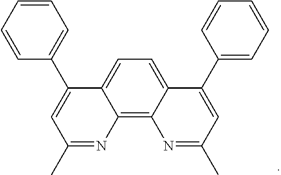 [SMILES: Cc1cc(-c2ccccc2)c2ccc3c(-c4ccccc4)cc(C)nc3c2n1]

The material of the electron injection layer 6 is formed by a compound having a structure below, blended with the electron injection material LiF:

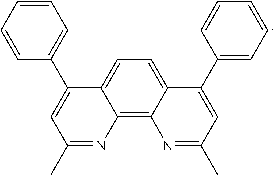 [SMILES: Cc1cc(-c2ccccc2)c2ccc3c(-c4ccccc4)cc(C)nc3c2n1]

In the organic light-emitting device, the light emitting layer 32 is formed by blending a host material and a guest luminescent dye, where the host material is the fused cyclic compound (D-2), and the guest material is the compound RD, and the host material and the guest material are blended at a weight ratio of 100:5. The organic light-emitting device is configured to have a particular structure of ITO/hole injection layer (HIL)/hole transport layer (HTL)/organic light emitting layer (fused cyclic compound D-3 blended with compound RD)/electron transport layer (ETL)/electron injection layer (EIL/LiF)/cathode (Al). The fused cyclic compound (D-3), and the compound RD have the following chemical structures:

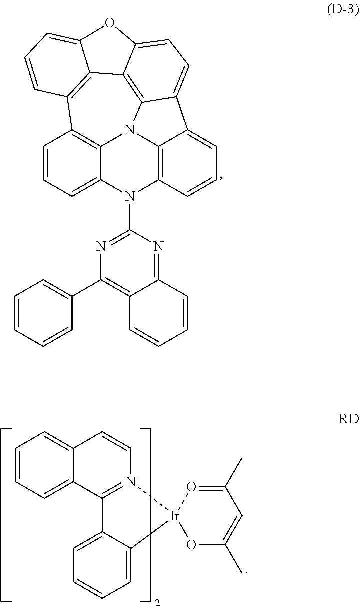

The host material in the light-emitting layer is the fused cyclic compound of Formula D-3, which has increased effective conjugation in the compound by the fusion mode of the phenyl ring and the heterocyclic ring in the mother nuclear structure, and facilitates the balancing of the electron transport performance while improving the hole performance of the compound. The fused cyclic compound of D-3 has high triplet energy level and glass transition temperature, which ensures the efficient transfer of energy from the host material to the guest material and prevents the crystallization of the material molecules of the light emitting layer. Meanwhile, the compound has ambipolarity, the HOMO level and the LUMO level of the host material are respectively located on different electron donating group

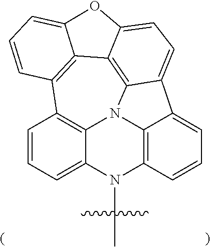 [SMILES: *N1c2cccc3c4cccc5oc6ccc7c8cccc1c8n(c23)c7c6c54]

and electron withdrawing group (quinazoline), and the transport of charges and holes in the host material becomes more balanced, thereby expanding the area where holes and electrons are recombined into excitons in the light emitting layer, reducing the exciton concentration, preventing the triplet-triplet annihilation of the device, and improving the efficiency of the device; and also the carrier recombination region in the host material is allowed to stay far away from the interface of the light emitting layer to the hole or electron transport layer, thus improving the color purity of the OLED device, avoiding the returning of excitons to the transport layer, and further improving the efficiency of the device.

The HOMO level and the LUMO level of the fused cyclic compound D-3 match those of the adjacent hole transport layer and electron transport layer, so that the OLED device has a small driving voltage.

The HOMO level and the LUMO level of the fused cyclic compound D-3 are relatively separated, with a small difference (ΔE_{ST}) between singlet and triplet energy levels, which promotes the reverse intersystem crossing of triplet excitons to singlet excitons. Moreover, the high reverse intersystem crossing (RISC) rate from the triplet state T1 to the singlet state S1 of the hot material can suppress the Dexter energy transfer (DET) from the host material to the luminescent dye, promote the FÖrster energy transfer, reduce the exciton loss generated during the Dexter energy transfer (DET), thus avoiding the efficiency roll-off of the organic light-emitting device and improving the luminescence efficiency of the device.

In an alternative embodiment, the host material in the light emitting layer may also be any fused cyclic compound of Formulas (D-1)-(D-24).

### Example 9

This example provides an organic light-emitting device, which differs from the organic light-emitting device provided in Embodiment 8 only in that the host material in the light emitting layer is a fused heterocyclic compound having a structure shown below:

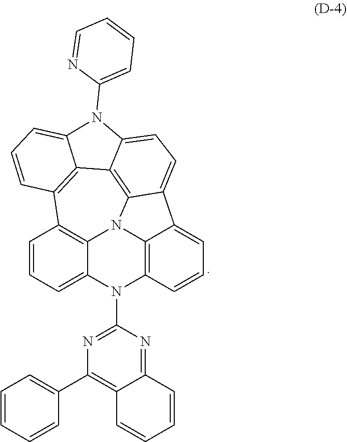 [SMILES: c1ccc(-c2nc(N3c4cccc5c6cccc7c6c6c(ccc8c9cccc3c9n(c45)c86)n7-c3ccccn3)nc3ccccc23)cc1]

### Example 10

This example provides an organic light-emitting device, which differs from the organic light-emitting device provided in Embodiment 8 only in that the host material in the light emitting layer is a fused heterocyclic compound having a structure shown below:

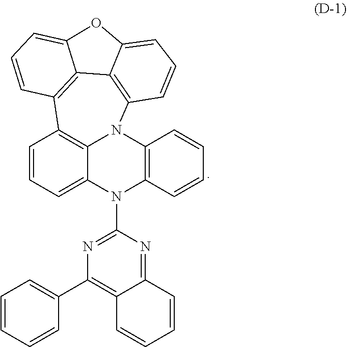 [SMILES: c1ccc(-c2nc(N3c4ccccc4-n4c5cccc6oc7cccc(c8cccc3c84)c7c65)nc3ccccc23)cc1]

### Example 11

This example provides an organic light-emitting device, which differs from the organic light-emitting device provided in Embodiment 8 only in that the host material in the light emitting layer is a fused heterocyclic compound having a structure shown below:

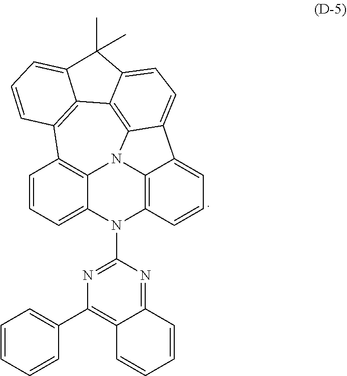 [SMILES: CC1(C)c2cccc3c2-c2c1ccc1c4cccc5c4n(c21)-c1c-3cccc1N5c1nc(-c2ccccc2)c2ccccc2n1]

### Example 12

This example provides an organic light-emitting device, which differs from the organic light-emitting device provided in Embodiment 8 only in that the host material in the light emitting layer is a fused heterocyclic compound having a structure shown below:

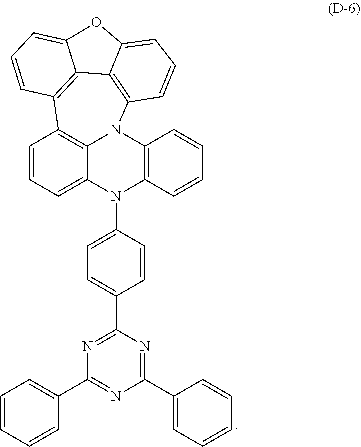 [SMILES: c1ccc(-c2nc(-c3ccccc3)nc(-c3ccc(N4c5ccccc5-n5c6cccc7oc8cccc(c9cccc4c95)c8c76)cc3)n2)cc1]

### Example 13

This example provides an organic light-emitting device, which differs from the organic light-emitting device provided in Embodiment 8 only in that the host material in the light emitting layer is a fused heterocyclic compound having a structure shown below:

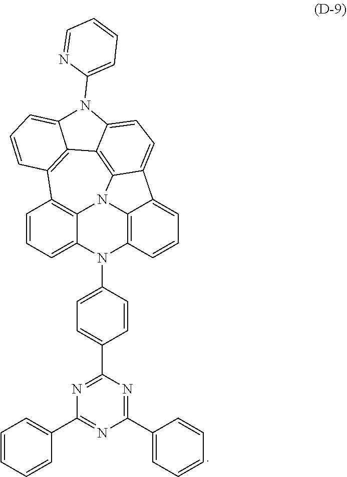 [SMILES: c1ccc(-c2nc(-c3ccccc3)nc(-c3ccc(N4c5cccc6c7cccc8c7c7c(ccc9c%10cccc4c%10n(c56)c97)n8-c4ccccn4)cc3)n2)cc1]

### Example 14

This example provides an organic light-emitting device, which differs from the organic light-emitting device provided in Embodiment 8 only in that the host material in the light emitting layer is a fused heterocyclic compound having a structure shown below:

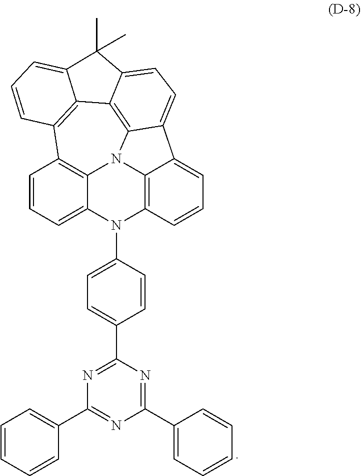 [SMILES: CC1(C)c2cccc3c2-c2c1ccc1c4cccc5c4n(c21)-c1c-3cccc1N5c1ccc(-c2nc(-c3ccccc3)nc(-c3ccccc3)n2)cc1]

### Comparative Example 1

This comparative example provides an organic light-emitting device, which differs from the organic light-emitting device provided in Embodiment 7 only in that the host material in the light emitting layer is 4,4′-bis(9-carbazole)biphenyl (CBP).

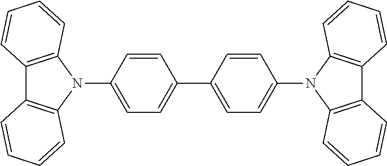 [SMILES: c1ccc2c(c1)c1ccccc1n2-c1ccc(-c2ccc(-n3c4ccccc4c4ccccc43)cc2)cc1]

### Test Example 1

1. Determination of Glass Transition Temperature

The glass transition temperature of the material according to the present invention was tested by differential scanning calorimetery (DSC) in a range from room temperature to 400° C. at a ramping rate of 10° C./min under nitrogen atmosphere.

2. The fluorescence and phosphorescence spectra of a solution of the fused heterocyclic compound (having a concentration of 10⁻⁵ mol/L) in toluene were measured at 298 K and 77 K, respectively, the corresponding singlet (S1) and triplet (T1) energy levels were calculated according to the formula E=1240/λ (S1), and then a difference between the singlet and triplet energy levels of the fused heterocyclic compound was obtained. The difference between the energy levels of the fused heterocyclic compound is shown below.

TABLE 1

| | Fused Heterocyclic Compound | | | | | | |
|---|---|---|---|---|---|---|---|
| | Formula D-3 | Formula D-4 | Formula D-1 | Formula D-5 | Formula D-6 | Formula D-9 | Formula D-8 |
| Glass | 170 | 172 | 168 | 169 | 170 | 175 | 171 |
| Transition | | | | | | | |
| Temperature | | | | | | | |
| (° C.) | | | | | | | |
| T1 (eV) | 2.68 | 2.63 | 2.71 | 2.64 | 2.70 | 2.64 | 2.62 |
| S1-T1 (eV) | 0.15 | 0.16 | 0.25 | 0.18 | 0.20 | 0.14 | 0.15 |

### Test Example 2

The current, voltage, brightness, and luminescence spectrum of the device were tested synchronously using PR 650 scanning spectroradiometer and Keithley K 2400 digital source meter. The organic light-emitting devices provided in Examples 8-14 and Comparative Example 1 were tested. The results are shown in Table 2.

TABLE 2

| | | | Current | Current | |
|---|---|---|---|---|---|
| | Host | | density/ | efficiency | Chromaticity |
| | material | Voltage/V | mA/cm² | cd/A | (CIE-X, Y) |
| Comparative | CBP | 5.1 | 10 | 21 | (0.66, 0.33) |
| Example 1 | | | | | |
| Example 8 | Formula | 3.9 | 10 | 27 | (0.66, 0.33) |
| | D-3 | | | | |
| Example 9 | Formula | 3.8 | 10 | 28 | (0.66, 0.33) |
| | D-4 | | | | |
| Example 10 | Formula | 3.9 | 10 | 27 | (0.66, 0.33) |
| | D-1 | | | | |
| Example 11 | Formula | 3.8 | 10 | 26 | (0.66, 0.33) |
| | D-5 | | | | |
| Example 12 | Formula | 3.7 | 10 | 24 | (0.66, 0.33) |
| | D-6 | | | | |
| Example 13 | Formula | 3.7 | 10 | 27 | (0.66, 0.33) |
| | D-9 | | | | |
| Example 14 | Formula | 3.6 | 10 | 25 | (0.66, 0.33) |
| | D-8 | | | | |

The organic light-emitting devices provided in Examples 8-14 and Comparative Example 1 were tested. The results are shown in Table 2. The luminescence efficiency of the OLED devices provided in Examples 8-14 is higher than that of the OLED device in Comparative Example 1, and the driving voltage is lower than that of the OLED device in Comparative Example 1, indicating that the fused heterocyclic compound provided in the present invention, when used as a host material in a light emitting layer of an OLED device, can effectively improve the luminescence efficiency and reduce the driving voltage of the device.

Apparently, the above-described embodiments are merely examples provided for clarity of description, and are not intended to limit the implementations of the present invention. Other variations or changes can be made by those skilled in the art based on the above description. The embodiments are not exhaustive herein. Obvious variations or changes derived therefrom also fall within the protection scope of the present invention.

What is claimed is:

1. A fused cyclic compound, having a structure of Formula (I) or (II): 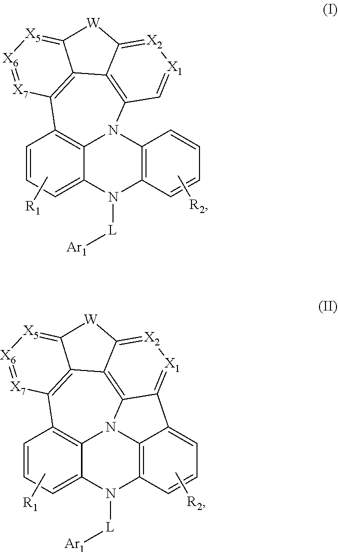
wherein W is selected from O, S, C—Ar₂ or N—Ar₂;
X₁ is selected from N or C—R₁ₐ, X₂ is selected from N or C—R₂ₐ, X₅ is selected from N or C—R₅ₐ, X₆ is selected from N or C—R₆ₐ, X₇ is selected from N or C—R₇ₐ, in which
R₁ₐ, R₂ₐ, R₅ₐ, R₆ₐ, and R₇ₐ are each independently selected from hydrogen, halo, cyano, alkyl, alkenyl, alkynyl, cycloalkyl, alkoxy, silyl, aryl or heteroaryl;
R₁, and R₂ are each independently selected from hydrogen, halo, cyano, alkyl, alkenyl, alkynyl, cycloalkyl, alkoxy, silyl, aryl or heteroaryl; L is a single bond, a C₁-C₁₀ substituted or unsubstituted aliphatic hydrocarbon group, a C₆-C₆₀ substituted or unsubstituted aryl group, or a C₃-C₃₀ substituted or unsubstituted heteroaryl group; Ar₁ and Ar₂ are each independently selected from hydrogen, halo, cyano, alkyl, alkenyl, alkynyl, cycloalkyl, alkoxy, silyl, aryl or heteroaryl, in which
the heteroaryl has at least one heteroatom independently selected from nitrogen, sulfur, oxygen, phosphorous, boron or silicon.

2. The fused cyclic compound according to claim 1, wherein
R₁ and R₂ are each independently selected from hydrogen, halo, cyano, a C₁-C₃₀ substituted or unsubstituted alkyl group, a C₂-C₃₀ substituted or unsubstituted alkenyl group, a C₂-C₃₀ substituted or unsubstituted alkynyl group, a C₃-C₃₀ substituted or unsubstituted cycloalkyl group, a C₁-C₃₀ substituted or unsubstituted alkoxy group, a C₁-C₃₀ substituted or unsubstituted silyl group, a C₆-C₆₀ substituted or unsubstituted aryl group, or a C₃-C₃₀ substituted or unsubstituted heteroaryl group;
Ar₁ and Ar₂ are each independently selected from hydrogen, halo, cyano, a C₁-C₃₀ substituted or unsubstituted alkyl group, a C₂-C₃₀ substituted or unsubstituted alkenyl group, a C₂-C₃₀ substituted or unsubstituted alkynyl group, a C₃-C₃₀ substituted or unsubstituted cycloalkyl group, a C₁-C₃₀ substituted or unsubstituted alkoxy group, a C₁-C₃₀ substituted or unsubstituted silyl group, a C₆-C₆₀ substituted or unsubstituted aryl group, or a C₃-C₃₀ substituted or unsubstituted heteroaryl group; and
R₁ₐ, R₂ₐ, R₅ₐ, R₆ₐ, and R₇ₐ are each independently selected from hydrogen, halo, cyano, a C₁-C₃₀ substituted or unsubstituted alkyl group, a C₂-C₃₀ substituted or unsubstituted alkenyl group, a C₂-C₃₀ substituted or unsubstituted alkynyl group, a C₃-C₃₀ substituted or unsubstituted cycloalkyl group, a C₁-C₃₀ substituted or unsubstituted alkoxy group, a C₁-C₃₀ substituted or unsubstituted silyl group, a C₆-C₆₀ substituted or unsubstituted aryl group, or a C₃-C₃₀ substituted or unsubstituted heteroaryl group.

3. The fused cyclic compound according to claim 1, wherein Ar₁ and Ar₂ are each independently selected from any of the following groups; and R₁, R₂, R₁ₐ, R₂ₐ, R₅ₐ, R₆ₐ, and R₇ₐ are each independently selected from hydrogen or any of the following groups: 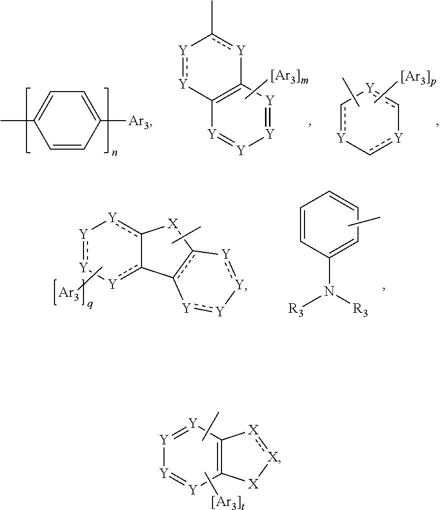
where X is nitrogen, oxygen or sulfur, Y is each independently nitrogen or carbon; and in the 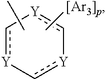
at least one Y is nitrogen; and
n is an integer of 0-5, m is an integer of 0-7, p is an integer of 0-6, q is an integer of 0-8, and t is an integer of 0-7; and [symbol] is a single or double bond;
R₃ is each independently selected from a substituted or unsubstituted phenyl group or hydrogen; and
Ar₃ is each independently selected from hydrogen, phenyl, coronenyl, pentalenyl, indenyl, naphthyl, azulenyl, fluorenyl, heptalenyl, octalenyl, benzodiindenyl, acenaphthylenyl, phenalenyl, phenanthrenyl, anthracenyl, triindenyl, fluoranthenyl, benzopyrenyl, benzoperylenyl, benzofluoranthenyl, acephenanthrenyl, aceanthrylenyl, 9,10-benzophenanthrenyl, pyrenyl, 1,2-benzophenanthrenyl, butylphenyl, naphthacenyl, pleiadenyl, picenyl, perylenyl, pentaphenyl, pentacenyl, tetraphenylene, cholanthrenyl, helicenyl, hexaphenyl, rubicenyl, coronenyl, trinaphthylenyl, heptaphenyl, pyranthrenyl, ovalenyl, corannulenyl, anthanthrenyl, truxenyl, pyranyl, benzopyranyl, furyl, benzofuryl, isobenzofuryl, oxanthracenyl, oxazolinyl, dibenzofuryl, peri-xanthenoxanthenyl, thienyl, thioxanthenyl, thianthrenyl, phenoxathiinyl, thionaphthenyl, isothionaphthenyl, thiophanthrenyl, dibenzothienyl, benzothienyl, pyrrolyl, pyrazolyl, tellurazolyl, selenazolyl, thiazolyl, isothiazolyl, oxazolyl, oxadiazolyl, furazanyl, pyridinyl, pyrazinyl, pyrimidinyl, pyridazinyl, triazinyl, indolizinyl, indolyl, isoindolyl, indazolyl, purinyl, quinolizinyl, isoquinolyl, carbazolyl, fluorenocarbazolyl, indolocarbazolyl, imidazolyl, naphthyridinyl, phthalazinyl, quinazolinyl, benzodiazepinyl, quinoxalinyl, cinnolinyl, quinolyl, pteridinyl, phenanthridinyl, acridinyl, perimidinyl, phenanthrolinyl, phenazinyl, carbolinyl, phenotellurazinyl, phenoselenazinyl, phenothiazinyl, phenoxazinyl, triphenodithiazinyl, azadibenzofuryl, triphenodioxazinyl, anthrazinyl, benzothiazolyl, benzoimidazolyl, benzoxazolyl, benzisoxazolyl or benzoisothiazolyl.

4. The fused cyclic compound according to claim 2, wherein Ar₁ and Ar₁ are each independently selected from any of the following groups; and R₁, R₂, R₁ₐ, R₂ₐ, R₅ₐ, R₆ₐ, and R₇ₐ are each independently selected from hydrogen or any of the following groups: 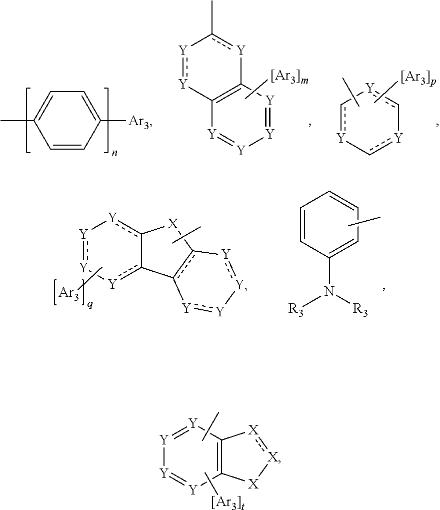
where X is nitrogen, oxygen or sulfur, Y is each independently nitrogen or carbon; and in the 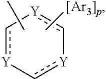
at least one Y is nitrogen; and
n is an integer of 0-5, m is an integer of 0-7, p is an integer of 0-6, q is an integer of 0-8, and t is an integer of 0-7; and [symbol] is a single or double bond;
R₃ is each independently selected from a substituted or unsubstituted phenyl group or hydrogen; and
Ar₃ is each independently selected from hydrogen, phenyl, coronenyl, pentalenyl, indenyl, naphthyl, azulenyl, fluorenyl, heptalenyl, octalenyl, benzodiindenyl, acenaphthylenyl, phenalenyl, phenanthrenyl, anthracenyl, triindenyl, fluoranthenyl, benzopyrenyl, benzoperylenyl, benzofluoranthenyl, acephenanthrenyl, aceanthrylenyl, 9,10-benzophenanthrenyl, pyrenyl, 1,2-benzophenanthrenyl, butylphenyl, naphthacenyl, pleiadenyl, picenyl, perylenyl, pentaphenyl, pentacenyl, tetraphenylene, cholanthrenyl, helicenyl, hexaphenyl, rubicenyl, coronenyl, trinaphthylenyl, heptaphenyl, pyranthrenyl, ovalenyl, corannulenyl, anthanthrenyl, truxenyl, pyranyl, benzopyranyl, furyl, benzofuryl, isobenzofuryl, oxanthracenyl, oxazolinyl, dibenzofuryl, peri-xanthenoxanthenyl, thienyl, thioxanthenyl, thianthrenyl, phenoxathiinyl, thionaphthenyl, isothionaphthenyl, thiophanthrenyl, dibenzothienyl, benzothienyl, pyrrolyl, pyrazolyl, tellurazolyl, selenazolyl, thiazolyl, isothiazolyl, oxazolyl, oxadiazolyl, furazanyl, pyridinyl, pyrazinyl, pyrimidinyl, pyridazinyl, triazinyl, indolizinyl, indolyl, isoindolyl, indazolyl, purinyl, quinolizinyl, isoquinolyl, carbazolyl, fluorenocarbazolyl, indolocarbazolyl, imidazolyl, naphthyridinyl, phthalazinyl, quinazolinyl, benzodiazepinyl, quinoxalinyl, cinnolinyl, quinolyl, pteridinyl, phenanthridinyl, acridinyl, perimidinyl, phenanthrolinyl, phenazinyl, carbolinyl, phenotellurazinyl, phenoselenazinyl, phenothiazinyl, phenoxazinyl, triphenodithiazinyl, azadibenzofuryl, triphenodioxazinyl, anthrazinyl, benzothiazolyl, benzoimidazolyl, benzoxazolyl, benzisoxazolyl or benzoisothiazolyl.

5. The fused cyclic compound according to claim 1, wherein R₁, R₂ and Ar₁ include at least one electron withdrawing group, and/or at least one electron donating group.

6. The fused cyclic compound according to claim 2, wherein R1, R2 and Ar1 include at least one electron withdrawing group, and/or at least one electron donating group.

7. The fused cyclic compound according to claim 3, wherein R1, R2 and Ar1 include at least one electron withdrawing group, and/or at least one electron donating group.

8. The fused cyclic compound according to claim 4, wherein R1, R2 and Ar1 include at least one electron withdrawing group, and/or at least one electron donating group.

9. The fused cyclic compound according to claim 1, having a molecular structure as shown below:

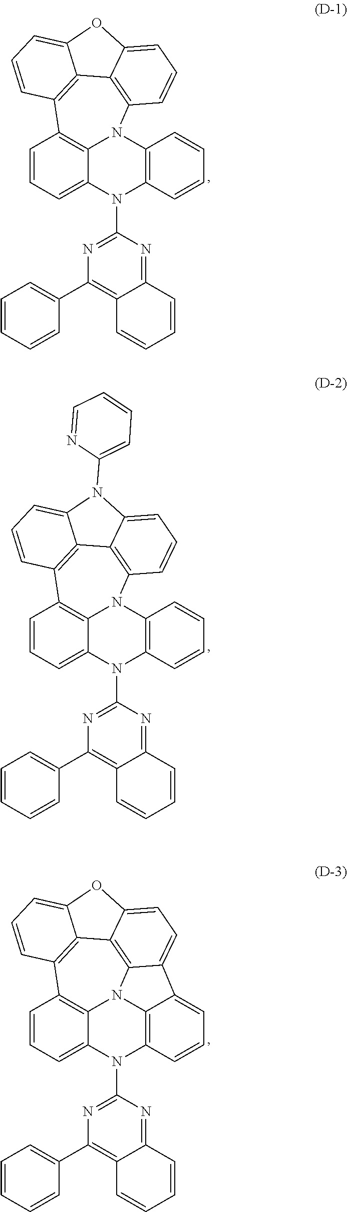 [SMILES: c1ccc(-c2nc(N3c4ccccc4-n4c5cccc6oc7cccc(c8cccc3c84)c7c65)nc3ccccc23)cc1 ; c1ccc(-c2nc(N3c4ccccc4-n4c5cccc6c5c5c(cccc5n6-c5ccccn5)c5cccc3c54)nc3ccccc23)cc1 ; c1ccc(-c2nc(N3c4cccc5c6cccc7oc8ccc9c%10cccc3c%10n(c45)c9c8c76)nc3ccccc23)cc1]

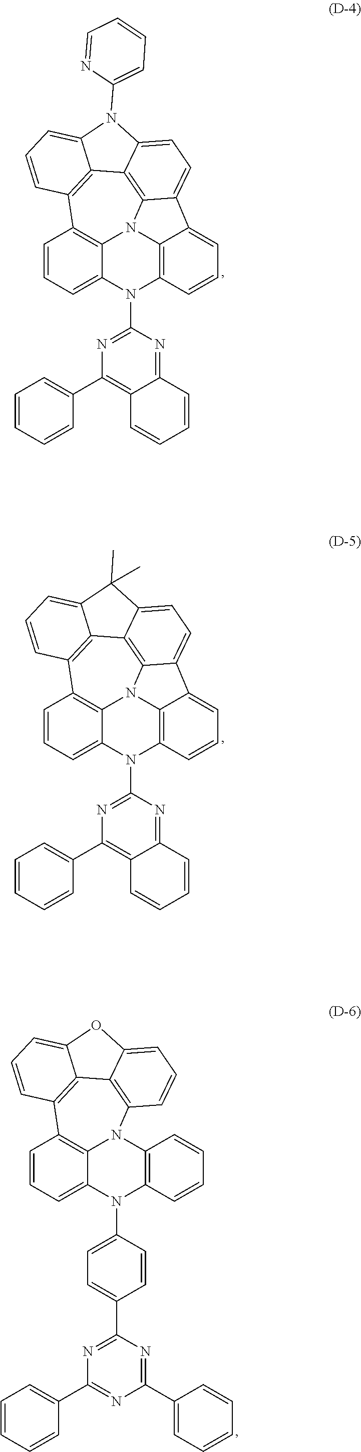 [SMILES: c1ccc(-c2nc(N3c4cccc5c6cccc7c6c6c(ccc8c9cccc3c9n(c45)c86)n7-c3ccccn3)nc3ccccc23)cc1 ; CC1(C)c2cccc3c2-c2c1ccc1c4cccc5c4n(c21)-c1c-3cccc1N5c1nc(-c2ccccc2)c2ccccc2n1 ; c1ccc(-c2nc(-c3ccccc3)nc(-c3ccc(N4c5ccccc5-n5c6cccc7oc8cccc(c9cccc4c95)c8c76)cc3)n2)cc1]

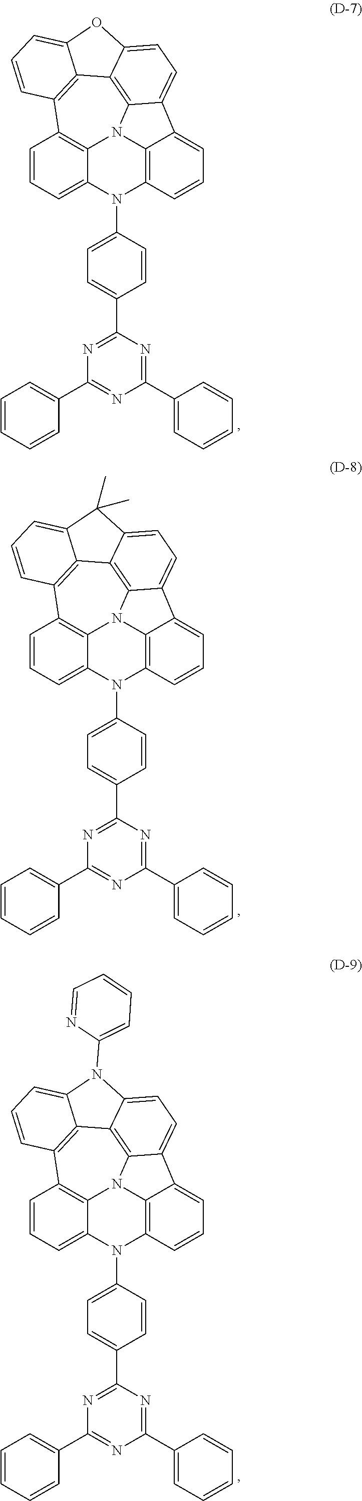 [SMILES: c1ccc(-c2nc(-c3ccccc3)nc(-c3ccc(N4c5cccc6c7cccc8oc9ccc%10c%11cccc4c%11n(c56)c%10c9c87)cc3)n2)cc1 ; CC1(C)c2cccc3c2-c2c1ccc1c4cccc5c4n(c21)-c1c-3cccc1N5c1ccc(-c2nc(-c3ccccc3)nc(-c3ccccc3)n2)cc1 ; c1ccc(-c2nc(-c3ccccc3)nc(-c3ccc(N4c5cccc6c7cccc8c7c7c(ccc9c%10cccc4c%10n(c56)c97)n8-c4ccccn4)cc3)n2)cc1]

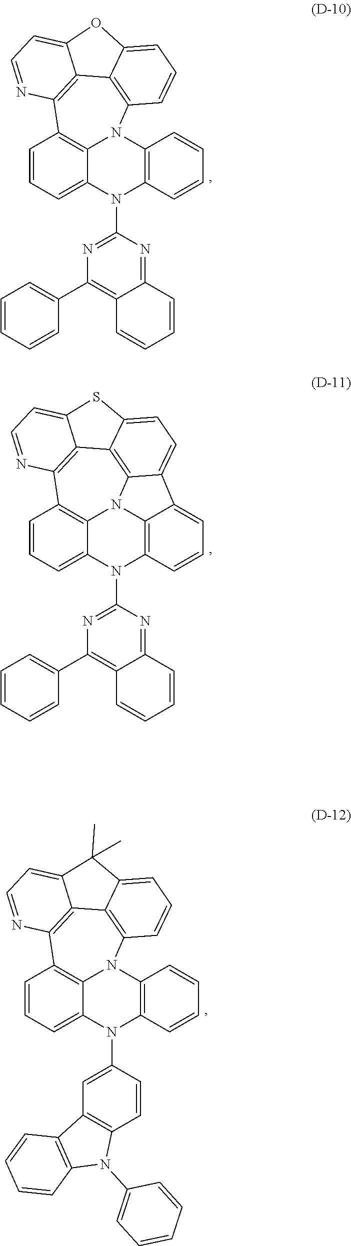 [SMILES: c1ccc(-c2nc(N3c4ccccc4-n4c5cccc6oc7ccnc(c8cccc3c84)c7c65)nc3ccccc23)cc1 ; c1ccc(-c2nc(N3c4cccc5c6ccc7sc8ccnc9c%10cccc3c%10n(c45)c6c7c89)nc3ccccc23)cc1 ; CC1(C)c2cccc3c2-c2c1ccnc2-c1cccc2c1N3c1ccccc1N2c1ccc2c(c1)c1ccccc1n2-c1ccccc1]

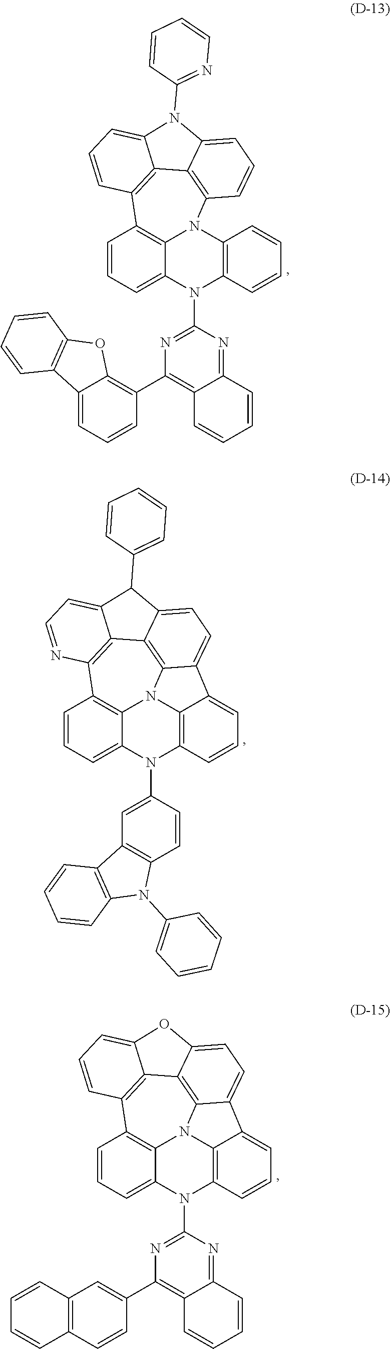 [SMILES: c1ccc(-n2c3cccc4c5cccc6c5n(c5cccc2c5c43)-c2ccccc2N6c2nc(-c3cccc4c3oc3ccccc34)c3ccccc3n2)nc1 ; c1ccc(C2c3ccnc4c3-c3c2ccc2c5cccc6c5n(c32)-c2c-4cccc2N6c2ccc3c(c2)c2ccccc2n3-c2ccccc2)cc1 ; c1ccc2cc(-c3nc(N4c5cccc6c7cccc8oc9ccc%10c%11cccc4c%11n(c56)c%10c9c87)nc4ccccc34)ccc2c1]

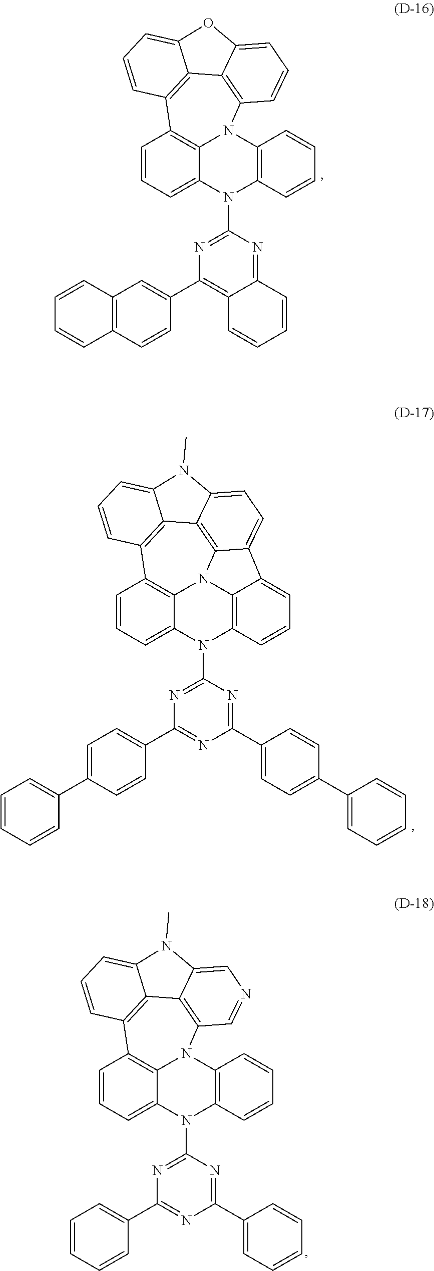 [SMILES: c1ccc2c(c1)N(c1nc(-c3ccc4ccccc4c3)c3ccccc3n1)c1cccc3c4cccc5oc6cccc(c6c54)n-2c13 ; Cn1c2cccc3c4cccc5c4n4c6c(cccc6c6ccc1c(c32)c64)N5c1nc(-c2ccc(-c3ccccc3)cc2)nc(-c2ccc(-c3ccccc3)cc2)n1 ; Cn1c2cccc3c4cccc5c4n(c4cncc1c4c32)-c1ccccc1N5c1nc(-c2ccccc2)nc(-c2ccccc2)n1]

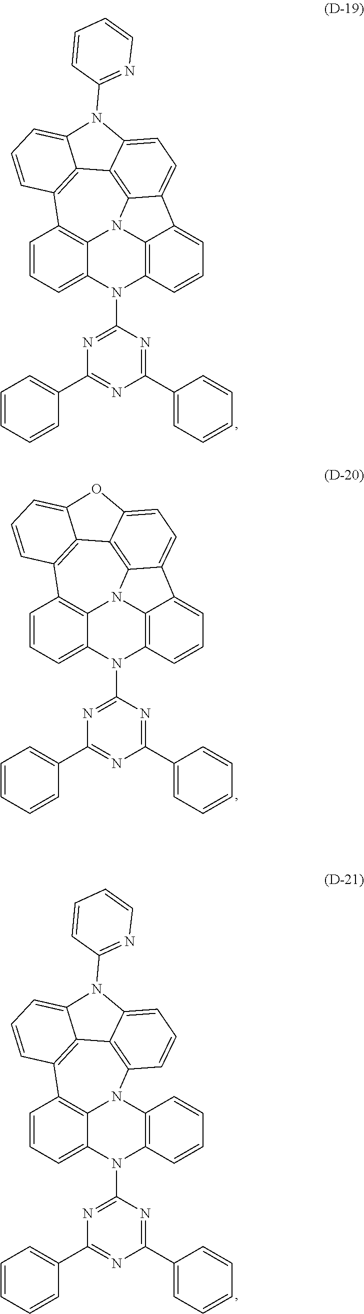 [SMILES: c1ccc(-c2nc(-c3ccccc3)nc(N3c4cccc5c6cccc7c6c6c(ccc8c9cccc3c9n(c45)c86)n7-c3ccccn3)n2)cc1 ; c1ccc(-c2nc(-c3ccccc3)nc(N3c4cccc5c6cccc7oc8ccc9c%10cccc3c%10n(c45)c9c8c76)n2)cc1 ; c1ccc(-c2nc(-c3ccccc3)nc(N3c4ccccc4-n4c5cccc6c5c5c(cccc5n6-c5ccccn5)c5cccc3c54)n2)cc1]

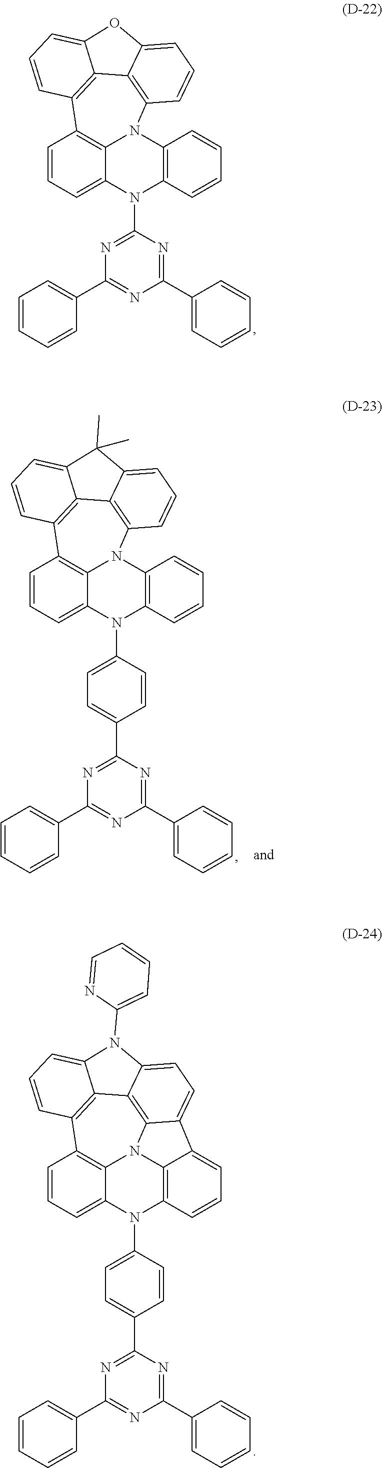 [SMILES: c1ccc(-c2nc(-c3ccccc3)nc(N3c4ccccc4-n4c5cccc6oc7cccc(c8cccc3c84)c7c65)n2)cc1 ; CC1(C)c2cccc3c2-c2c(cccc21)N1c2ccccc2N(c2ccc(-c4nc(-c5ccccc5)nc(-c5ccccc5)n4)cc2)c2cccc-3c21 ; c1ccc(-c2nc(-c3ccccc3)nc(-c3ccc(N4c5cccc6c7cccc8c7c7c(ccc9c%10cccc4c%10n(c56)c97)n8-c4ccccn4)cc3)n2)cc1]

10. The fused cyclic compound according to claim 1, wherein the fused cyclic compound is an organic electroluminescent material.

11. The fused cyclic compound according to claim 2, wherein the fused cyclic compound is an organic electroluminescent material.

12. The fused cyclic compound according to claim 3, wherein the fused cyclic compound is an organic electroluminescent material.

13. The fused cyclic compound according to claim 4, wherein the fused cyclic compound is an organic electroluminescent material.

14. The fused cyclic compound according to claim 5, wherein the fused cyclic compound is an organic electroluminescent material.

15. The fused cyclic compound according to claim 6, wherein the fused cyclic compound is an organic electroluminescent material.

16. The fused cyclic compound according to claim 7, wherein the fused cyclic compound is an organic electroluminescent material.

17. A method for preparing a fused cyclic compound according to claim 1, wherein
the compound of Formula (I) is synthesized through the following steps:
subjecting a compound of Formula (A) and a compound of Formula (B) as starting materials to a coupling reaction in the presence of a catalyst, to obtain an intermediate 1;
cyclizing the intermediate 1, to obtain an intermediate 2; and subjecting the intermediate 2 and a compound T₃-L-Ar₁ to a substitution or coupling reaction in the presence of a catalyst, to obtain the compound of Formula (I),
where the synthesis route for the compound of Formula (I) is shown below: 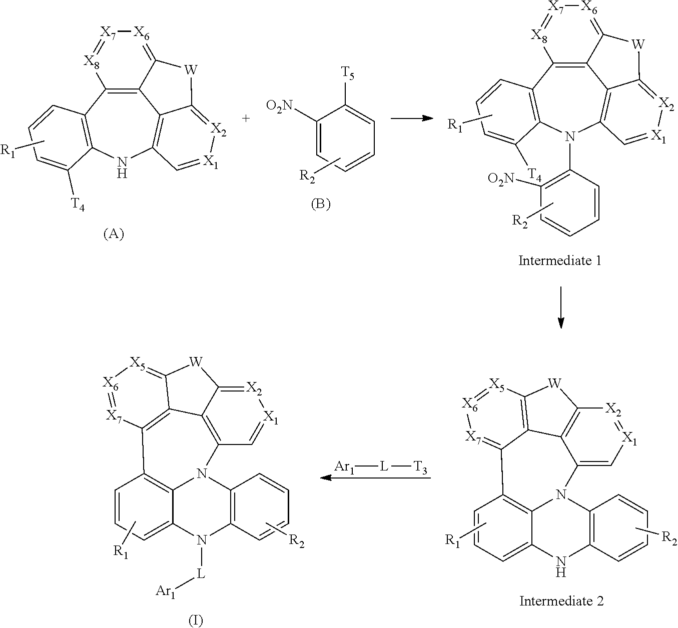
the compound of Formula (II) is synthesized through the following steps:
subjecting a compound of Formula (C) and a compound of Formula (E) as starting materials to a coupling reaction in the presence of a catalyst, to obtain an intermediate 3; cyclizing the intermediate 3, to obtain an intermediate 4; reducing the nitro group of the intermediate 4 and then subjecting the intermediate 4 to a coupling reaction, to obtain an intermediate 5; and subjecting the intermediate 5 and a compound T₃-L-Ar₁ to a substitution or coupling reaction in the presence of a catalyst, to obtain the compound of Formula (II),
the synthesis route for the compound of Formula (II) is shown below: 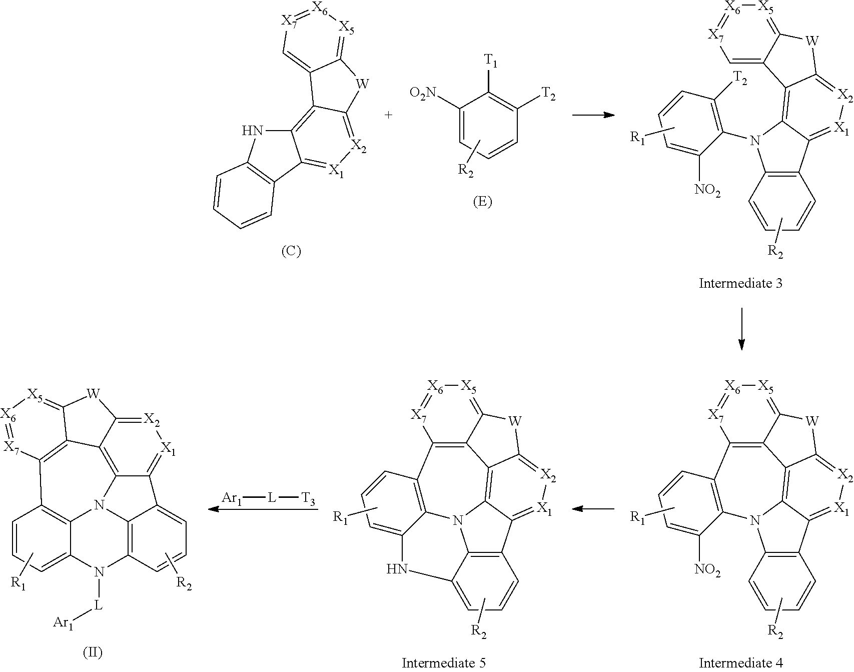
where T₁-T₅ are each independently selected from hydrogen, fluoro, chloro, bromo or iodo.

18. An organic light-emitting device, having at least one functional layer containing a fused cyclic compound according to claim 1.

19. The organic light-emitting device according to claim 18, wherein the functional layer is a light emitting layer.

20. The organic light-emitting device according to claim 19, wherein the material of the light emitting layer comprises a host material and a guest luminescent dye, where the host material is the fused cyclic compound.
